(19) United States
(12) Patent Application Publication
(10) Pub. No.: US 2024/0390057 A1
(43) Pub. Date: Nov. 28, 2024

(54) PULSED FIELD ABLATION APPARATUS AND RELATED METHODS

(71) Applicant: AtriCure, Inc., Mason, OH (US)

(72) Inventors: Matthew Monti, Cincinnati, OH (US); Jackson Thomas Romelli, Cincinnati, OH (US); Dominic James Armagno, Crestwood, KY (US); Paighton Anne Schrupp, St. Louis Park, MN (US); Salvatore Privitera, Mason, OH (US); Adam Harp, Cincinnati, OH (US); Kenneth Lance Miller, Hamilton, OH (US)

(73) Assignee: AtriCure, Inc., Mason, OH (US)

(21) Appl. No.: 18/672,173

(22) Filed: May 23, 2024

Related U.S. Application Data
(60) Provisional application No. 63/504,210, filed on May 25, 2023.
(60) Provisional application No. 63/506,898, filed on Jun. 8, 2023.
(60) Provisional application No. 63/506,900, filed on Jun. 8, 2023.

(51) Int. Cl. A61B 18/14; A61B 18/00; A61B 18/02; A61B 18/12

(52) U.S. Cl. CPC A61B 18/14; A61B 18/02; A61B 18/1206; A61B 2018/00083; A61B 2018/00136; A61B 2018/00357; A61B 2018/00363; A61B 2018/00577; A61B 2018/00613; A61B 2018/00642; A61B 2018/00732; A61B 2018/00761; A61B 2018/00767; A61B 2018/00839; A61B 2018/00994; A61B 2018/0225; A61B 2018/1253; A61B 2018/126; A61B 2018/1435; A61B 2018/1467

(57) ABSTRACT

A pulsed field ablation effector comprising: (a) an electrode including an electrode surface for delivering electric current to anatomical tissue, and a deformable insulator selectively covering the electrode surface, the deformable insulator configured to deform when contacted by the anatomical tissue to expose the electrode surface. Also disclosed are methods and devices for carrying out electroporation and other forms of ablation concomitant with electroporation.

### CROSS-REFERENCE TO RELATED APPLICATIONS

The present application claims the benefit of U.S. Provisional Patent Application No. 63/504,210, filed on May 25, 2023 and titled “PULSED FIELD ABLATION APPARATUS AND RELATED METHODS, U.S. Provisional Patent Application No. 63/506,898, filed on Jun. 8, 2023 and titled “PULSED FIELD ABLATION APPARATUS AND RELATED METHODS, and U.S. Provisional Patent Application No. 63/506,900, filed on Jun. 8, 2023 and titled “PULSED FIELD ABLATION APPARATUS AND RELATED METHODS, the disclosure of each of which is hereby incorporated by reference in its entirety.

### INTRODUCTION TO THE INVENTION

The present disclosure is directed to ablation devices, and, more specifically, to ablation systems and devices configured to perform pulsed field ablation, components thereof, and related methods.

The present disclosure contemplates that ablation systems configured to perform pulsed field ablation (“PFA”) may be used in various medical and surgical procedures. Generally, PFA systems may be used to ablate targeted cells while limiting potential collateral damage to non-targeted tissues. PFA typically involves applying high-voltage electrical pulses to a target tissue. The pulses create high-intensity electrical fields, which disrupt the integrity of the cell membranes in the target tissue. As a result, over a short period of time (e.g., days to weeks), the cells die, creating a lesion in the target tissue.

The present disclosure contemplates that PFA may be used for ablation of cardiac tissue for treatment of cardiac arrhythmias. Some known PFA technologies, such as catheter-based devices, may produce sub-optimal results in some circumstances. For example, the present disclosure contemplates that it may be difficult to maintain desired contact pressure between catheter-based devices and an interior heart wall, thus potentially resulting in lesions that may be inaccurately positioned and/or less durable than desired.

While known PFA systems have been used to perform some cardiac ablation procedures, particularly endocardial ablations (e.g., on the inside surface of the heart), improvements in the construction and operation of PFA systems and PFA devices may be beneficial for users (e.g., physicians and surgeons) and patients. The present disclosure includes various improvements that may enhance the construction, operation, and use of PFA systems and PFA devices, including embodiments applicable to epicardial ablation (e.g., on the outside surface of the heart and/or penetrating the tissue surface).

It is a first aspect of the present invention to provide a pulsed field ablation effector comprising: (a) an electrode including an electrode surface for delivering electric current to anatomical tissue; and, (b) a deformable insulator selectively covering the electrode surface, the deformable insulator configured to deform when contacted by the anatomical tissue to expose the electrode surface.

In a more detailed embodiment of the first aspect, the deformable insulator includes a slit at least partially occupied by the electrode. In yet another more detailed embodiment, the slit longitudinally extends along a dominant dimension of the deformable insulator, and the electrode longitudinally extends within the slit for a majority of its length. In a further detailed embodiment, the pulsed field ablation effector further includes a rigid backer to which the electrode and deformable insulator are mounted. In still a further detailed embodiment, the deformable insulator is mounted to the rigid backer using a living hinge. In a more detailed embodiment, the deformable insulator is embedded within the rigid backer. In a more detailed embodiment, the deformable insulator includes a raised feature configured to concentrate contact force from contact with the anatomical tissue and hasten deformation of the deformable insulator. In another more detailed embodiment, the raised feature comprises a plurality of raised features, with at least two of the plurality of raised feature being on opposite sides of the electrode. In yet another more detailed embodiment, the plurality of raised features includes longitudinal ribs, and the longitudinal ribs extend generally parallel to the electrode.

In yet another more detailed embodiment of the first aspect, the deformable insulator comprises an elastomer. In yet another more detailed embodiment, the elastomer includes silicone. In a further detailed embodiment, the electrode is segmented into a plurality of electrodes, the deformable insulator is segmented into a plurality of deformable insulator sections, the plurality of electrodes each include electrode surfaces that are selectively covered by at least one of the plurality of deformable insulator sections, and only those of the plurality of deformable insulator sections contacted by the anatomical tissue deform to expose those of the plurality of electrodes covered by the plurality of deformable insulator sections contacted. In still a further detailed embodiment, the deformable insulator is segmented into a plurality of deformable insulator sections, the electrode surface is selectively covered by at least one of the plurality of deformable insulator sections, and only those of the plurality of deformable insulator sections contacted by the anatomical tissue deform to expose those aspects of the electrode surface covered by the plurality of deformable insulator sections contacted. In a more detailed embodiment, the pulsed field ablation effector further includes a cryogenic conduit configured to supply cryogenic fluid to a cryogenic tissue contact. In a more detailed embodiment, the cryogenic tissue contact comprises the electrode surface of the electrode. In another more detailed embodiment, the pulse field ablation effector further includes a radio frequency electrode adapted to deliver radio frequency energy to the anatomical tissue. In yet another more detailed embodiment, the radio frequency electrode is selectively covered by the deformable insulator.

It is a second aspect of the present invention to provide a method of performing a pulsed field tissue ablation, the method comprising: (a) repositioning a pulsed field ablation effector into proximity with a target tissue, where the pulsed field ablation effector includes an electrode having an ablation surface covered by a deformable insulator; (b) repositioning the pulsed field ablation effector to sufficiently contact the target tissue, where sufficient contact with the target tissue is operative to deform the deformable insulator and expose an ablation surface of the electrode that was previously covered by the deformable insulator; (c) supplying electric current to the electrode, when in sufficient contact with the target tissue, to cause electroporation to the target tissue; and, (d) repositioning the pulsed field ablation effector to no longer sufficiently contact the target tissue, where insufficient contact with the target tissue is operative to deform the deformable insulator and cover the ablation surface of the electrode that was previously uncovered.

In a more detailed embodiment of the second aspect, the target tissue is cardiac tissue, and the contact is epicardial contact. In yet another more detailed embodiment, the target tissue is a nerve, and the contact is at least one of with an intact nerve and a dissected nerve. In a further detailed embodiment, the target tissue is an intercostal nerve, and the method is performed concomitant with a thoracotomy. In still a further detailed embodiment, the target tissue is cardiac tissue, and the contact is endocardial contact. In a more detailed embodiment, the pulsed field ablation effector includes a first jaw and a second jaw, where the electrode includes a first electrode portion on the first jaw and a second electrode portion on the second jaw, and repositioning the pulsed field ablation effector to sufficiently contact the target tissue includes bringing the first electrode portion into contact with epicardial cardiac tissue, and bringing the second electrode portion into contact with endocardial cardiac tissue, so that sufficient contact with the epicardial cardiac tissue and endocardial cardiac tissue is operative to deform the deformable insulator and expose the first and second electrode portions previously covered by the deformable insulator. In a more detailed embodiment, the method further includes conducting a cryoablation concomitant with the electroporation to cause destruction of the target tissue or tissue in proximity thereto. In another more detailed embodiment, the method further includes conducting a radio frequency ablation concomitant with the electroporation to cause destruction of the target tissue or tissue in proximity thereto.

In yet another more detailed embodiment of the second aspect, the deformable insulator interposes the ablation surface of the electrode and the target tissue in the absence of sufficiently contact between the target tissue and the deformable insulator, and the deformable insulator no longer interposes the ablation surface of the electrode and the target tissue when sufficient contact occurs between the target tissue and the deformable insulator. In yet another more detailed embodiment, the tissue contacting surface of the electrode erupts from within the deformable insulator upon sufficient contact between the target tissue and the deformable insulator. In a further detailed embodiment, the electrode is segmented into a plurality of electrodes, each of the plurality of electrodes having a tissue contacting surface, the deformable insulator is segmented into a plurality of deformable insulator sections, with each of the plurality of electrodes being selectively covered by at least one of the plurality of deformable insulator sections, and each of the plurality of deformable insulator sections is operative to expose the corresponding tissue contacting surface of the plurality of electrodes when sufficient contact occurs between the target tissue and each of the plurality of deformable insulator sections. In still a further detailed embodiment, the deformable insulator is segmented into a plurality of deformable insulator sections with the ablation surface being selectively covered by at least one of the plurality of deformable insulator sections, the deformable insulator interposes the ablation surface of the electrode and the target tissue in the absence of sufficiently contact between the target tissue and the deformable insulator, and each of the plurality of deformable insulator sections is operative to expose a portion of the ablation surface when sufficient contact occurs between the target tissue and each of the plurality of deformable insulator sections.

It is a third aspect of the present invention to provide a method of inhibiting unintended arcing across a pulsed field ablation electrode, the method comprising covering the pulsed field ablation electrode with a deformable insulator, the deformable insulator configured to change its shape between a first shape and a second shape responsive to a sufficient external force being applied thereto, the first shape covering a tissue contacting surface of the pulsed field ablation electrode, and the second shape uncovering the tissue contacting surface of the pulsed field ablation electrode.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements may be exaggerated relative to other elements. Embodiments incorporating teachings of the present disclosure are shown and described with respect to the figures presented herein.

FIG. 1 is a simplified schematic diagram of an example PFA system 100, according to at least some aspects of the present disclosure.

FIG. 2 is a perspective view of an example clamp-type PFA device 200, according to at least some aspects of the present disclosure.

FIG. 3A is a perspective view of an example minimally invasive PFA device 300, according to at least some aspects of the present disclosure.

FIG. 3B is a simplified schematic view of the PFA device 300 of FIG. 3A.

FIG. 4A is a perspective view of an example needle-type PFA device 400, according to at least some aspects of the present disclosure.

FIG. 4B is a perspective view of the example needle-type PFA device 400 of FIG. 4A.

FIG. 4C is a magnified perspective view of a distal end of the example needle-type PFA device 400 of FIG. 4A.

FIG. 5A illustrates an elongated, generally linear electrode, according to at least some aspects of the present disclosure.

FIG. 5B illustrates a point electrode, according to at least some aspects of the present disclosure.

FIG. 5C illustrates a segmented electrode, according to at least some aspects of the present disclosure.

FIG. 5D illustrates an example nested electrode arrangement, according to at least some aspects of the present disclosure.

FIG. 5E illustrates an example squiggle electrode, according to at least some aspects of the present disclosure.

FIG. 5F illustrates an example perpendicular electrode arrangement, according to at least some aspects of the present disclosure.

FIG. 5G illustrates an example parallel electrode arrangement, according to at least some aspects of the present disclosure.

FIG. 5H illustrates an example continuous electrode, according to at least some aspects of the present disclosure.

FIG. 5I illustrates two example electrode arrays, according to at least some aspects of the present disclosure.

FIG. 5J illustrates example plate electrodes, according to at least some aspects of the present disclosure.

FIG. 5K illustrates an example electrode configuration including multiple pairs of cooperating electrodes, according to at least some aspects of the present disclosure.

FIG. 5L illustrates example raised electrodes, according to at least some aspects of the present disclosure.

FIG. 5M illustrates an example electrode configuration including raised electrodes positioned opposite a plate electrode, according to at least some aspects of the present disclosure.

FIG. 5N is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5O is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5P is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5Q is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5R is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5S is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 5T is a simplified section view of an alternative example electrode configuration that may be used, for example, in connection with PFA devices generally similar to PFA devices shown in FIGS. 2, 3A, 4A, and 22, according to at least some aspects of the present disclosure.

FIG. 6A is a simplified section view of a vacuum-clamp configuration, according to at least some aspects of the present disclosure.

FIG. 6B illustrates a baseline example embodiment including substantially flat opposed tissue engagement surfaces, according to at least some aspects of the present disclosure.

FIG. 6C illustrates an example embodiment including opposed convex tissue engagement surfaces, according to at least some aspects of the present disclosure.

FIG. 6D illustrates an example embodiment including opposed concave tissue engagement surfaces, according to at least some aspects of the present disclosure.

FIG. 6E illustrates an example embodiment including a convex tissue engagement surface opposing a cooperating concave tissue engagement surface, according to at least some aspects of the present disclosure.

FIG. 7 is a graphical illustration of the composition of an example multiple burst PFA signal, according to at least some aspects of the present disclosure.

FIG. 8 is a table listing example PFA signal parameters that may be used in connection with a variety of PFA devices, according to at least some aspects of the present disclosure.

FIG. 9 is a cross-sectional view showing electrode placing with respect to one another and on opposites of tissue as part of explaining bipolar/monopolar configurations and biphasic/monophasic signals, according to at least some aspects of the present disclosure.

FIG. 10A is a plot of an example ECG trace, according to at least some aspects of the present disclosure.

FIG. 10B illustrates two example embodiments configured to mechanically measure the distance between opposed jaws, according to at least some aspects of the present disclosure.

FIG. 10C illustrates four example embodiments configured to electrically and/or electronically measure the distance between opposed jaws, according to at least some aspects of the present disclosure.

FIG. 10D illustrates an example ratcheting clamp mechanism, according to at least some aspects of the present disclosure.

FIG. 11A is a perspective view of an example insulator configuration comprising a compressible insulator at least partially circumscribing one or more electrodes, according to at least some aspects of the present disclosure.

FIG. 11B is a different perspective view of the example insulator configuration depicted in FIG. 11A.

FIG. 11C is a simplified section view of the embodiment of FIG. 11A.

FIG. 12A is a perspective view of an example insulator configuration forming a shortened electrode exposure region, according to at least some aspects of the present disclosure.

FIG. 12B is a simplified section view of the embodiment of FIG. 12A.

FIG. 12C is another simplified section view of the embodiment of FIG. 12A.

FIG. 13A is a perspective view of an example configuration comprising an insulated jaw, according to at least some aspects of the present disclosure.

FIG. 13B is a section view of the embodiment of FIG. 13A.

FIG. 13C is a simplified perspective view of an alternative embodiment in which an electrode is selectively insulated, according to at least some aspects of the present disclosure.

FIG. 14A is a perspective view of an example configuration comprising a jaw with selectively insulated electrodes, according to at least some aspects of the present disclosure.

FIG. 14B is a section view of the embodiment of FIG. 14A.

FIG. 14C is another section view of the embodiment of FIG. 14A.

FIG. 15A is a perspective view of an alternative example configuration comprising a jaw with selectively insulated electrodes, according to at least some aspects of the present disclosure.

FIG. 15B is a section view of the embodiment of FIG. 15A.

FIG. 15C is another section view of the embodiment of FIG. 15A.

FIG. 16A is a perspective view of an alternative example configuration comprising a jaw with selectively insulated electrodes, according to at least some aspects of the present disclosure.

FIG. 16B is a section view of the embodiment of FIG. 16A.

FIG. 16C is another section view of the embodiment of FIG. 16A.

FIG. 17A is a perspective view of an alternative example configuration comprising a jaw with selectively insulated electrodes, according to at least some aspects of the present disclosure.

FIG. 17B is a section view of the embodiment of FIG. 17A.

FIG. 17C is another section view of the embodiment of FIG. 17A.

FIG. 18A is a perspective view of an alternative example configuration comprising a jaw with selectively insulated electrodes, according to at least some aspects of the present disclosure.

FIG. 18B is a section view of the embodiment of FIG. 18A.

FIG. 18C is another section view of the embodiment of FIG. 18A.

FIG. 18D illustrates an example embodiment including a deformable insulator disposed about an electrode disposed on a rigid back plate, according to at least some aspects of the present disclosure.

FIG. 18E illustrates an example embodiment including spaced-apart posts supporting an electrode within a deformable insulator, according to at least some aspects of the present disclosure.

FIG. 18F illustrates an example embodiment including raised features of a deformable insulator configured to contact tissue and expose an electrode, according to at least some aspects of the present disclosure.

FIG. 18G illustrates an alternative example embodiment in which a deformable insulator may be segmented, according to at least some aspects of the present disclosure.

FIG. 19A is a top view of an example lesion including a PFA zone and a thermal ablation zone, according to at least some aspects of the present disclosure.

FIG. 19B is a section view of the lesion of FIG. 19A.

FIG. 19C is a top view of an example lesion created using a PFA and RF device, depicting a PFA zone and a thermal ablation zone, according to at least some aspects of the present disclosure.

FIG. 19D illustrates an example snare clamp, according to at least some aspects of the present disclosure.

FIG. 19E illustrates an example generally helical screw engagement element for a jaw or electrode configured to penetrate the target tissue, according to at least some aspects of the present disclosure.

FIG. 20 is a simplified lateral view of an example PFA device including an expandable structure, according to at least some aspects of the present disclosure.

FIG. 21 is a simplified block diagram of an example equipment configuration, which may be used, for example, for using various PFA and/or RF ablation devices and/or algorithms according to at least some aspects of the present disclosure.

FIG. 22 is a perspective view of an example minimally invasive PFA device, according to at least some aspects of the present disclosure.

FIG. 23 is a table listing exemplary parameters or settings for operating a PFA device, according to at least some aspects of the present disclosure.

### DETAILED DESCRIPTION

Example embodiments according to the present disclosure are described and illustrated below to encompass devices, methods, and techniques relating to PFA. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure. Unless explicitly stated otherwise, any feature or function described in connection with any example embodiment may apply to any other example embodiment, and repeated description of similar features and functions is omitted for brevity.

The present disclosure contemplates that PFA may kill cells by using high-intensity electrical fields to cause irreversible nanopore formation in cell membranes, which is known as irreversible electroporation (“IRE”). IRE may be used to create deep and uniform lesions in cardiac tissue, which can be useful for treating arrythmias. If the electrical signal applied to the target tissue has insufficient intensity to cause IRE, reversible electroporation may occur. Pores formed by reversible electroporation may not be permanent, and affected cells typically recover after a short period of time (e.g., hours, days, weeks). The present disclosure contemplates that the minimum electric field strength (or voltage) required to cause IRE may depend on the characteristics of the electrical signal that is applied to the target tissue and the target tissue itself. For example, the number of pulses, frequency, magnitude, duration, shape, etc., may affect the extent of electroporation. In some circumstances, a region of IRE may be at least partially bounded by a region of reversible electroporation.

For context, an example PFA protocol configured to cause IRE may include a series of energy pulses (i.e., 100 Volts direct current (VDC)) at a given duration (i.e., 100 microseconds (s)) at a given frequency (i.e., 0.1 Hz to 10 Hz). For this sort of protocol, an electric field is applied to the tissue to create a cellular transmembrane voltage potential that may be in the range from 0.5 kV/cm to 2.5 kV/cm, depending on the tissue and how tissue damage is assessed. In some circumstances, electroporation efficacy may not be directly related to the amount of delivered energy or the charge. For example, in some circumstances, two 100 μs pulses of 1000 V/cm may be more effective for producing IRE than a single pulse of 200 μs with similar energy and charge. Additional details and alternatives are described elsewhere herein.

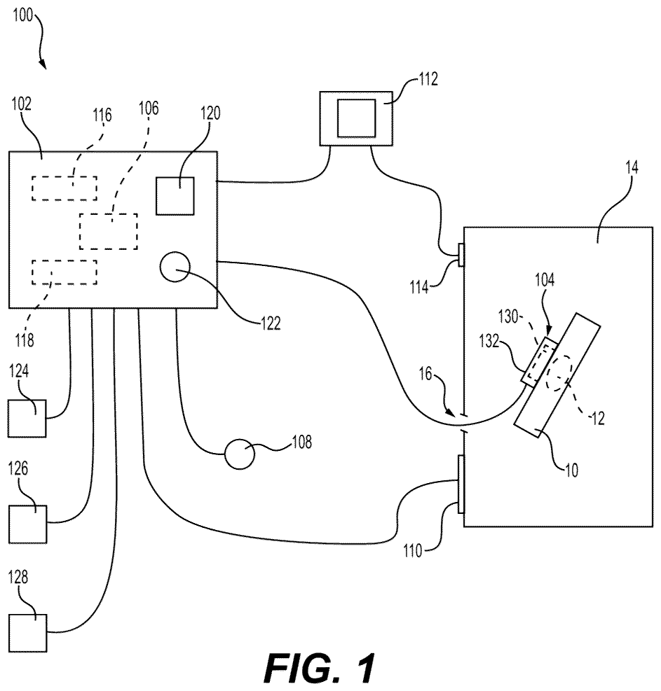

The following description of example embodiments with reference to FIGS. 1, 2, 3A, 3B, and 4A-4C provides context for various example apparatus features and methods described in more detail elsewhere herein. It is to be understood that any of these example embodiments may be utilized in connection with any feature or aspect described elsewhere herein.

Turning to FIG. 1, an example PFA system 100 may include a PFA unit 102, which may be operatively coupled to a PFA device 104. The PFA unit 102 may include a PFA generator 106, which may be configured to produce and/or supply electrical pulses for PFA. The PFA device 104 may be configured to apply the PFA pulses to a target tissue 10 in connection with creating a lesion 12 therein. In some example embodiments, the PFA unit 102 may be provided as capital (e.g., reusable) equipment and/or the PFA device 104 may be provided as disposable (e.g., single-use) equipment.

In some example embodiments, the target tissue 10 may be located internally within a patient's body 14. The PFA device 104 may be positioned proximate the target tissue 10 via any suitable patient access 16, such as arterial or venous access, percutaneous access, open surgical access, and/or minimally invasive surgical access. For example, in connection with treating cardiac arrhythmias, the target tissue 10 may include the heart wall (e.g., myocardium). In some example embodiments, the PFA device 104 may be positioned generally against the external (e.g., epicardial) surface of the heart wall and/or generally against the internal (e.g., endocardial) surface of the heart wall.

In some example embodiments, the PFA unit 102 may include and/or may be used in connection with various other components. For example, in some example embodiments, a foot switch 108 may be used to activate certain functions associated with the PFA unit 102, such as delivery of ablation energy to the PFA device 104. In some example embodiments, a return electrode 110 may be electrically coupled to the patient's body 14, such as to provide a return path for monopolar ablation energy delivered via the PFA device 104.

In some example embodiments, an electrocardiogram (“ECG”) monitor 112 may be used to display and/or analyze electrical impulses associated with the patient's heartbeat using one or more ECG electrodes 114. In some example embodiments, the ECG monitor 112 may be operatively coupled to and/or incorporated within the PFA unit 102, such as to facilitate synchronization of ablation pulse timing with the patient's heartbeat, as described below.

In some example embodiments, the PFA unit 102 may be configured to provide only PFA energy. In some example embodiments, the PFA unit 102 may be configured for use in connection with additional ablation modalities. For example, the PFA unit 102 may include and/or may be used in connection with one or more components configured for RF ablation, such as an RF generator 116, which may be generally similar to the “Ablation Sensing Unit (ASU),” “Ablation Switch Box (ASB),” and/or “Estech Electrosurgical unit (ESU)” available from AtriCure, Inc. of Mason, Ohio. As another example, the PFA unit 102 may include and/or may be used in connection with one or more components configured for cryosurgical ablation, such as a cryosurgical unit 118, which may be generally similar to the “cryoICE BOX” cryogenic surgical unit available from AtriCure, Inc. of Mason, Ohio. Generally, a particular lesion (or portion thereof) may be formed using PFA, one or more other ablation modalities, or any combination thereof, in any order sequentially and/or simultaneously (e.g., PFA and/or RF and/or cryo).

In some example embodiments, the PFA unit 102 may include one or more indicators and/or displays 120, which may provide information to the operator about the patient, the PFA unit 102, and/or an ablation. For example, some PFA units 102 may include integrated tissue interrogation/mapping functionality (such as voltage mapping, impedance mapping, exit/entrance block testing of lesions by cardiac pacing and sensing), which may use one or more dedicated electrodes and/or one or more electrodes associated with the PFA device 104. In some example embodiments, the PFA unit 102 may include one or more input devices 122, such as knobs, dials, switches, buttons, touch screens, etc., which may allow an operator to direct operation of various components of the PFA unit 102.

In some example embodiments, the PFA unit 102 may be configured with one or more external connections. For example, the PFA unit 102 may be operatively coupled to an electrical power source 124, such as a wall outlet. Some example embodiments may be operatively coupled to a vacuum source 126, such as an operating room vacuum system. Some example embodiments may be operatively coupled to a gas source 128, such as a compressed gas cylinder, which may contain a cryogenic fluid, for example.

In some example embodiments, the PFA device 104 may include one or more electrodes 130, which may be disposed in or on an end effector 132, to deliver PFA energy to the target tissue 10.

The description herein references a distal direction 18 and a proximal direction 20. The proximal direction 20 may be generally opposite the distal direction 18. As used herein, “distal” may refer to a direction generally away from an operator of a system or device (e.g., a surgeon), such as toward the distant-most end of a device that is inserted into a patient's body. As used herein, “proximal” may refer to a direction generally toward an operator of a system or device (e.g., a surgeon), such as away from the distant-most end of a device that is inserted into a patient's body. It is to be understood, however, that example directions referenced herein are merely for purposes of explanation and clarity, and should not be considered limiting.

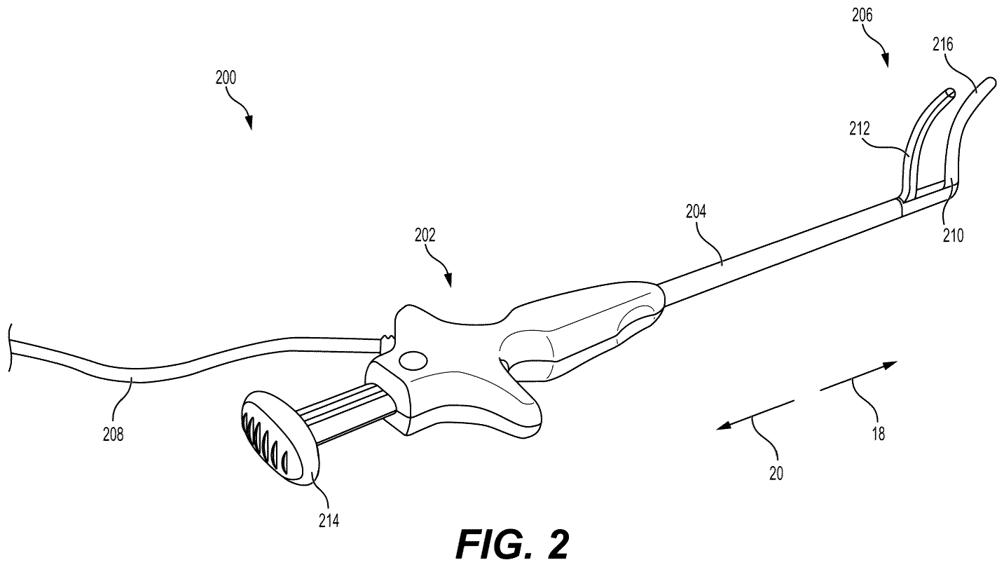

Referring to FIGS. 1 and 2, the illustrated clamp-type PFA device 200 may include a proximally disposed handle 202, a shaft 204 extending distally from the handle 202, and/or an end effector 206 disposed distally on the shaft 204. Generally, some example PFA devices 200 may be similar to the “Isolator Synergy” surgical ablation device available from AtriCure, Inc. of Mason, Ohio, and/or the devices described in U.S. Pat. No. 9,072,518, issued Jul. 7, 2015, titled “HIGH-VOLTAGE PULSE ABLATION SYSTEMS AND METHODS,” which is incorporated herein by reference in its entirety. Further, various PFA devices of any configuration and according to at least some aspects of the present disclosure may employ overlapping fields (e.g., focused between paired electrodes), on/off duty cycles (e.g., for thermal management), and/or constant signal generation switched to multiple electrode pairs generally similar to those utilized by the “Isolator Synergy” device and/or as described in U.S. Pat. No. 9,072,518.

Generally, any handle described herein with reference to any exemplary embodiment may be configured to be grasped by a human user (e.g., surgeon) and/or engaged by a non-human, mechanical and/or robotic device (e.g., a surgical robot). More generally, any handle described herein may comprise any structure that may be configured to be secured, held, and/or manipulated to position and/or restrain a PFA device, regardless of whether it may be held by a human (e.g., surgeon or assistant), robot, mechanical device, etc.

In the illustrated embodiment, a proximally extending connecting element 208 may electrically couple the PFA device 200 to the PFA unit 102. In some embodiments utilizing vacuum and/or cryogenics, the connecting element 208 may include suitable conduits. The end effector 206, corresponding to end effector 132, may comprise a distal repositionable or fixed jaw 210 and/or a movable proximal jaw 212. A plunger 214 or other actuator, which may be disposed proximally on the handle 202, may allow the operator to reposition one or both jaw 210, 212 to clamp the target tissue 10 between the jaws 210, 212. In the illustrated embodiment, one or both of the jaws 210, 212 may include one or more electrodes 216, corresponding to electrode 130, which may be utilized to deliver PFA energy to the target tissue 10. In embodiments including one or more electrodes, the electrode(s) may be positioned on either or both jaws 210, 212.

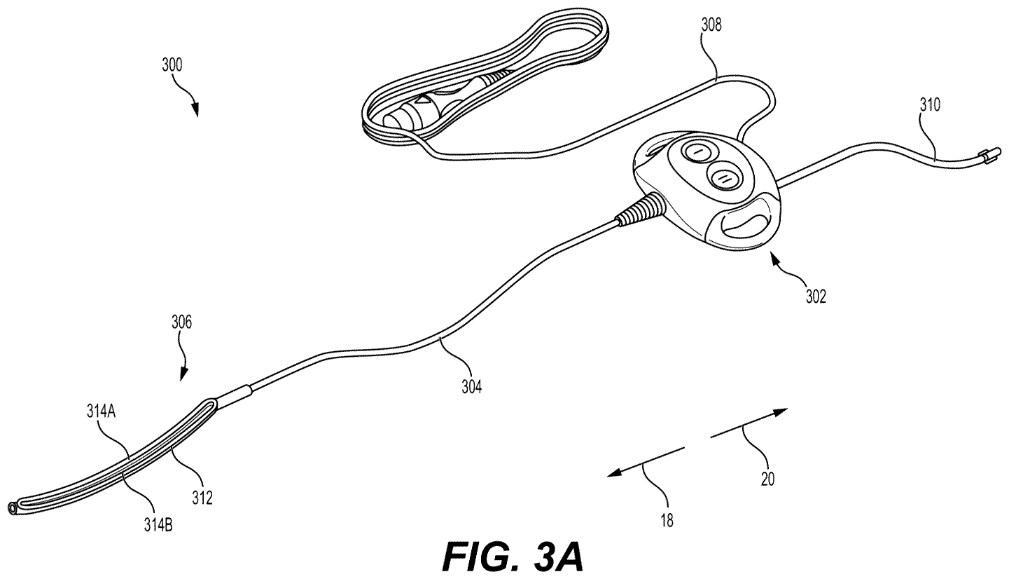

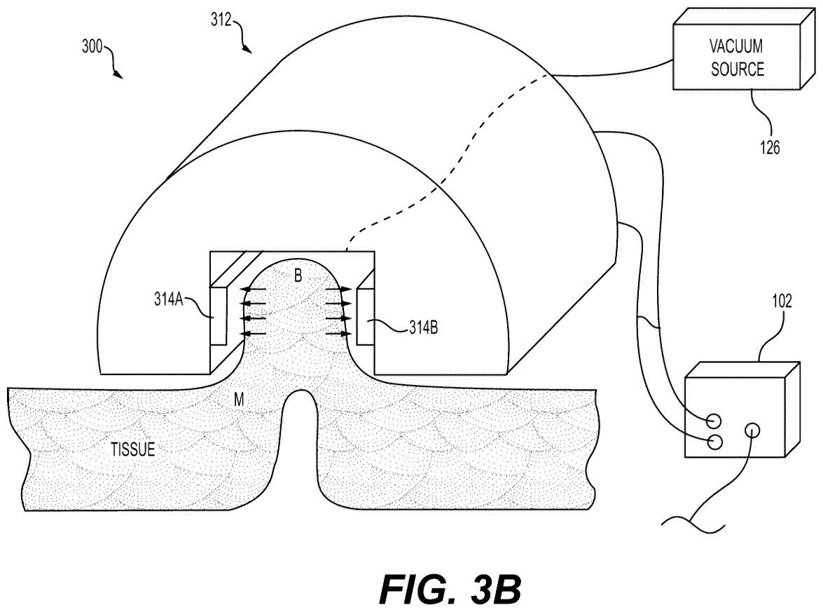

Referring to FIGS. 1, 3A, and 3B, the illustrated minimally invasive PFA device 300 may include a proximally disposed handle 302, a flexible connecting element 304 extending distally from the handle 302, and/or an end effector 306 disposed distally on the connecting element 304. Generally, some example PFA devices 300 may be similar to the “COBRA Fusion” ablation system available from AtriCure, Inc. of Mason, Ohio, and/or the devices described in U.S. Pat. No. 9,474,574, issued Oct. 25, 2016, titled “STABILIZED ABLATION SYSTEMS AND METHODS,” which is incorporated herein by reference in its entirety.

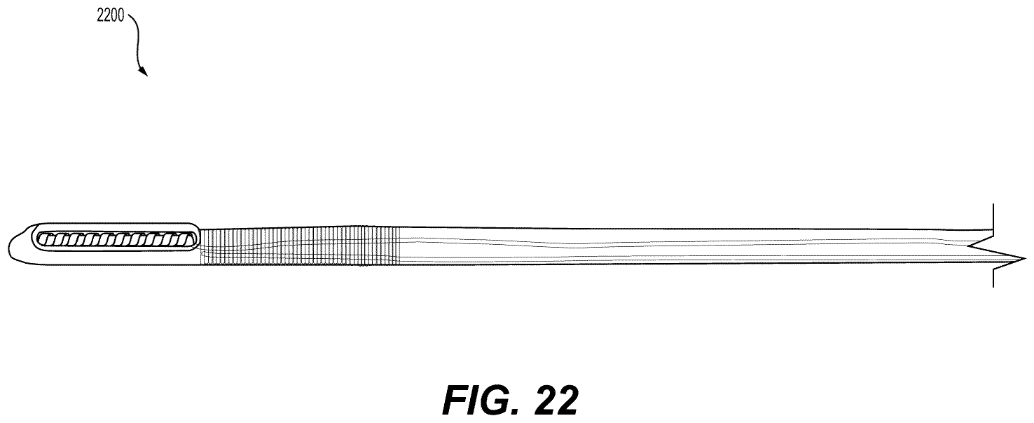

In the illustrated embodiment, a proximally extending electrical connecting element 308 may electrically couple the PFA device 300 to the PFA unit 102. A proximally extending vacuum connecting element 310 may fluidically couple the PFA device 300 to the PFA unit 102 and/or to the vacuum source 126. In the illustrated embodiment, the end effector 306, corresponding to end effector 132, may comprise an elongated, flexible stabilizer 312 configured to releasably engage the target tissue 10, such as by using vacuum. In the illustrated embodiment, one or more electrodes 314A, 314B, corresponding to electrode 130, may be disposed within the stabilizer 312 and/or may be utilized to deliver PFA energy to the target tissue 10. In some example embodiments, the PFA device 300 may be configured for vacuum-stabilized, unidirectional, and/or bipolar (or selectively bipolar/monopolar) operation. In some example embodiments, the target tissue may be folded, as generally shown in FIG. 3B. In some embodiments, the target tissue may be drawn into contact with the electrodes 314A, 314B without substantial tissue folding. The PFA device 2200 illustrated in FIG. 22 may be utilized in a similar manner.

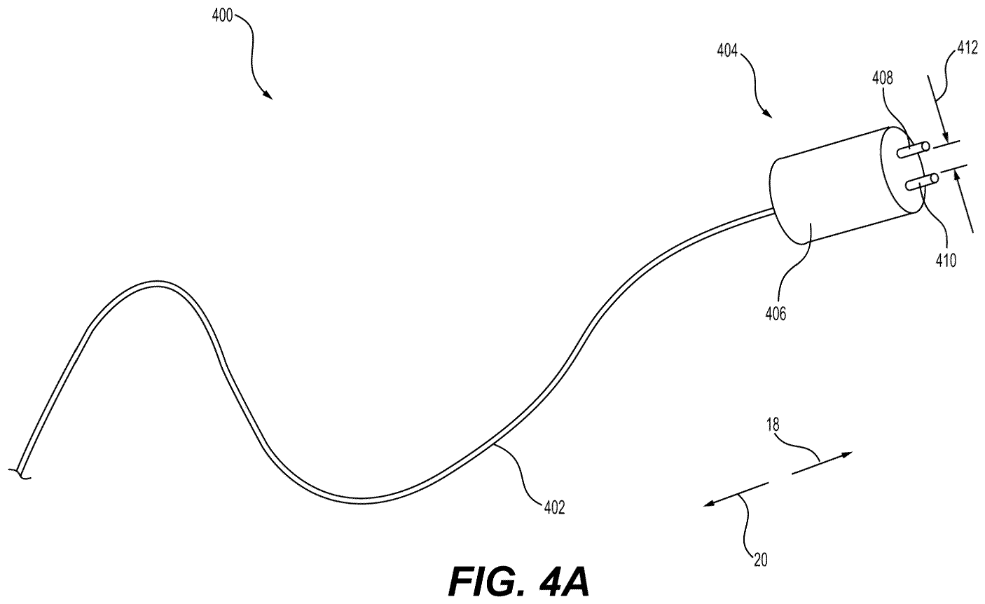

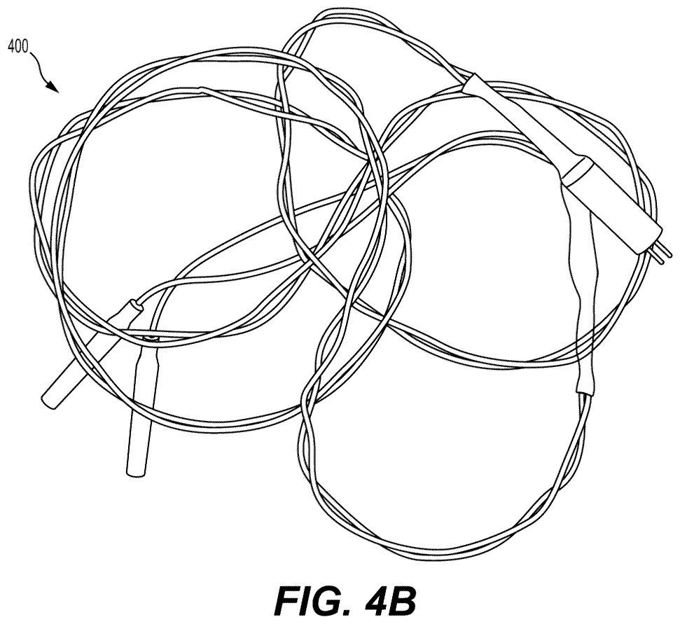

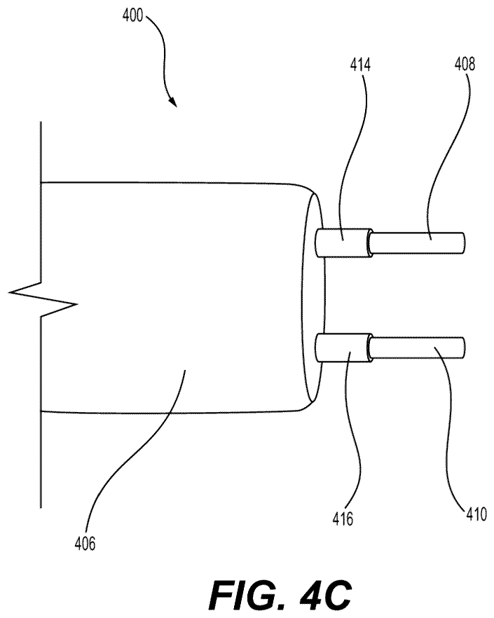

Referring to FIGS. 1 and 4A-4C, the illustrated needle-type PFA device 400 may include an elongated flexible connecting element 402 and/or an end effector 404 disposed distally on the connecting element 402. The connecting element 402 may electrically couple the PFA device 400 to the PFA unit 102. In the illustrated embodiment, the end effector 404, corresponding to end effector 132, may comprise a generally rigid housing 406 and/or one or more electrodes in the form of outwardly extending needles or pins 408, 410 configured to engage the target tissue 10, such as by making a depression therein or penetrating the target tissue 10. In some example embodiments, such penetration may provide desired tissue contact. The electrodes 408, 410, corresponding to electrode 130, may be utilized to deliver PFA energy to the target tissue 10. In the illustrated embodiment, the electrodes are spaced apart at a fixed, inter-electrode spacing 412. In some example embodiments, at least a portion of at least one pin 408, 410 may be covered by an insulator 414, 416, such as a proximal portion of one or more pins 408, 410. In various example embodiments, one or more pins 408, 410 may be generally blunt-tipped and/or generally sharply pointed. Example pin profiles may include short (shallow depth) pins and/or elongated (deep depth) pins, and combinations thereof. In some example embodiments, one or more pins 408, 410 may be in the form of a hollow needle configured to inject a substance into the target tissue. In some such embodiments, a substance may be injected into the target tissue, then PFA energy may be delivered to the target tissue via the needle operating as an electrode.

In view of the context provided by the example embodiments of FIGS. 1, 2, 3A, 3B, and 4A-4C, descriptions of various optional and alternative aspects and features follows.

Generally, PFA devices may be configured for unidirectional and/or bidirectional operation. As used herein, “unidirectional” may refer generally to application of PFA energy to a tissue from one side of the tissue. For example, applying PFA energy to only the epicardial surface of the heart, while not applying PFA energy to the opposed endocardial surface, is an example of unidirectional operation. As used herein, “bidirectional” may refer to application of PFA energy to a tissue from two opposed aspects so that the PFA energy flows through the tissue.

Example unidirectional devices may include needle-type PFA devices, pen-like PFA devices configured to create spot and/or linear lesions, endocardial catheter PFA devices, some minimally invasive, epicardial PFA devices, and/or surface-based end effectors including a plurality of electrodes operated at predetermined, different voltages. Some clamp-type devices, such as those utilizing electrodes on only one jaw, may have a unidirectional configuration.

Example bidirectional devices may include clamp-type PFA devices, graspers, some minimally invasive, epicardial PFA devices, and/or systems configured to place cooperating electrodes on opposing sides of tissue, such as the endocardial and epicardial surfaces of the heart (e.g., using magnetic coupling), or on anterior and posterior surfaces of bodily conduits. Example clamp-type PFA devices may be configured as pinch clamps or no-pinch clamps, and/or may be configured to substantially encircle an anatomic structure (e.g., pulmonary veins) or configured to ablate the wall of hollow organs via insertion of one jaw into a surgical purse string.

Some PFA devices, such as those described above with reference to FIGS. 1, 2, 3A, 3B, and 4A-4C, may include a variety of electrode configurations. Generally, any combination or variation of electrode configurations described herein may be used in connection with any embodiment according to at least some aspects of the present disclosure.

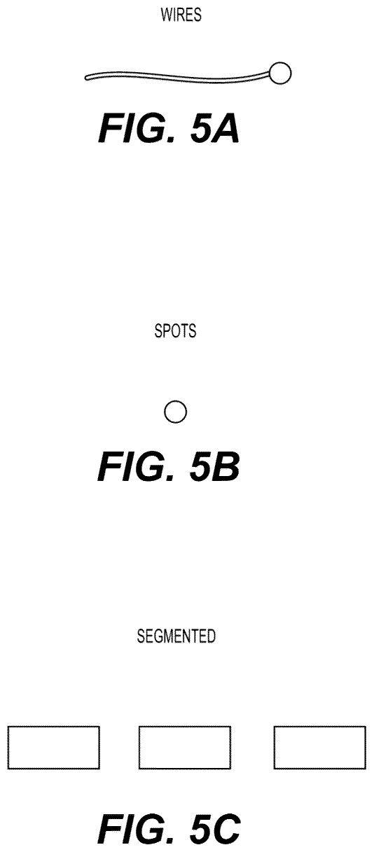

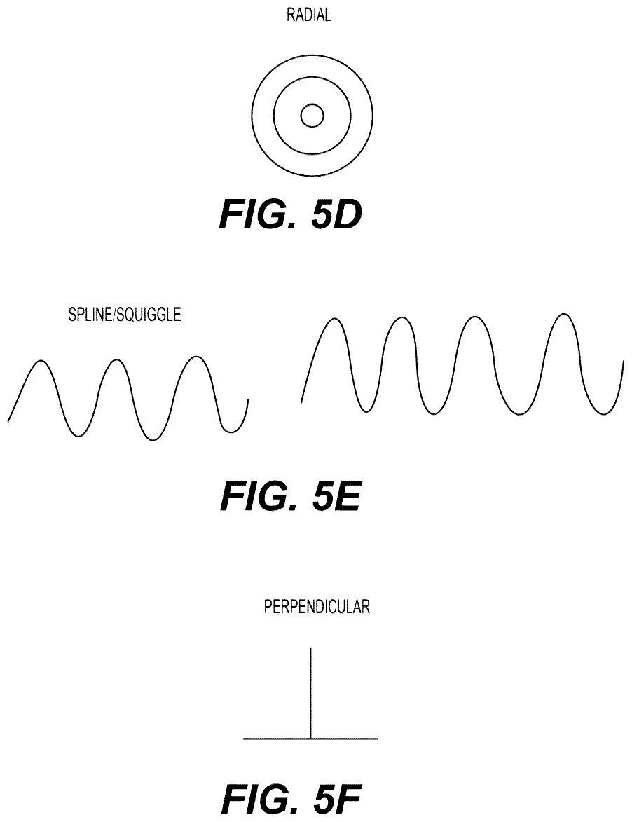

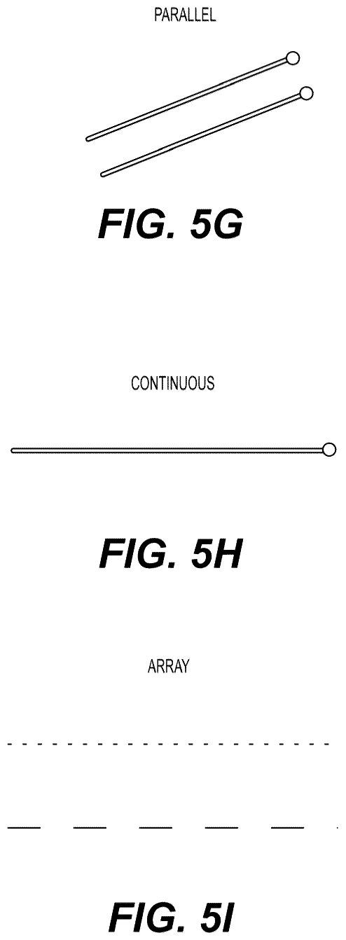

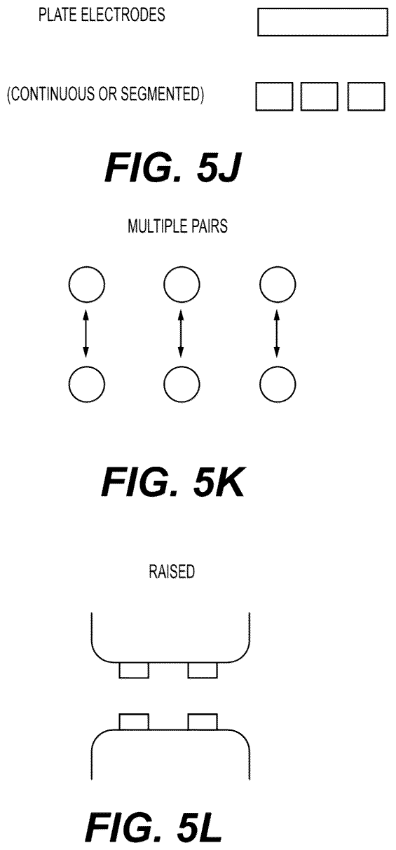

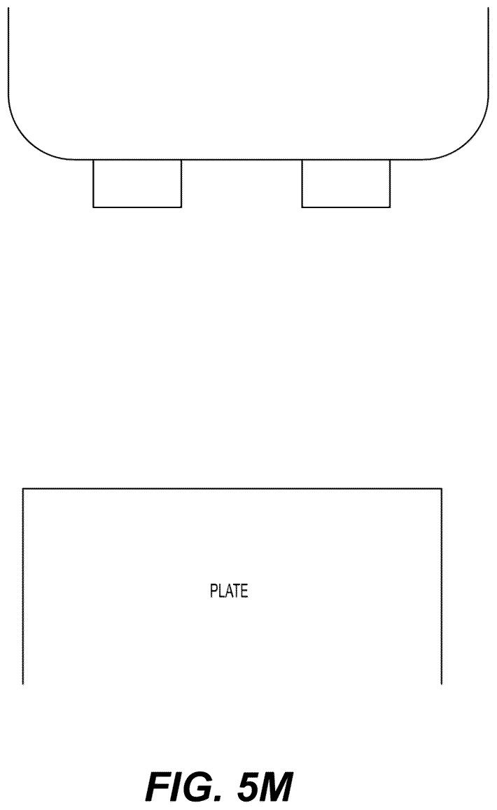

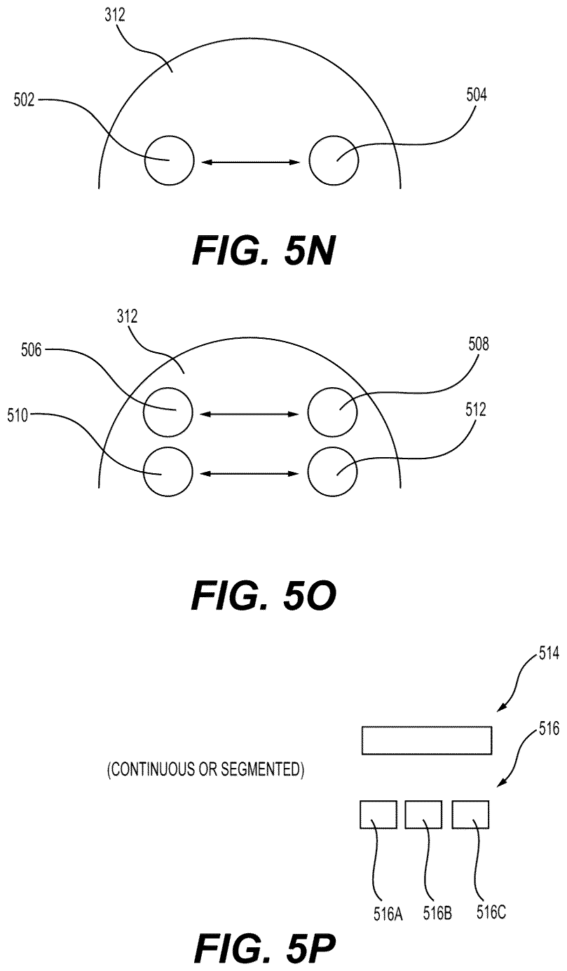

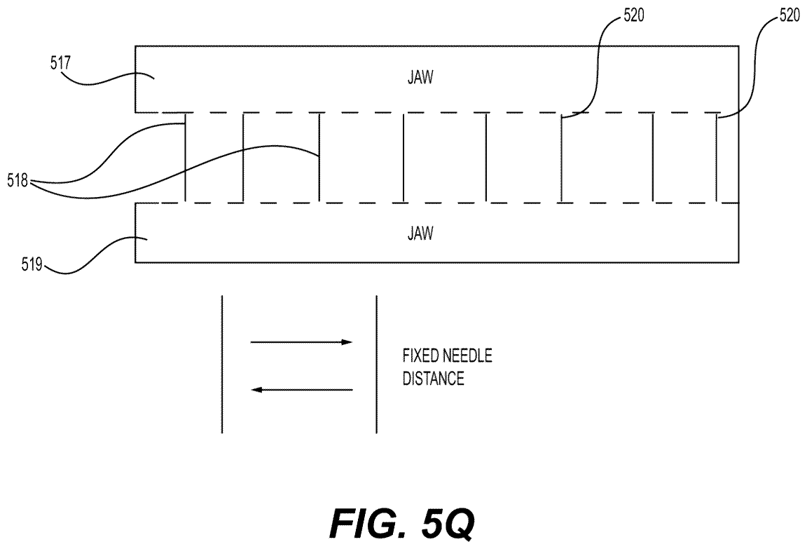

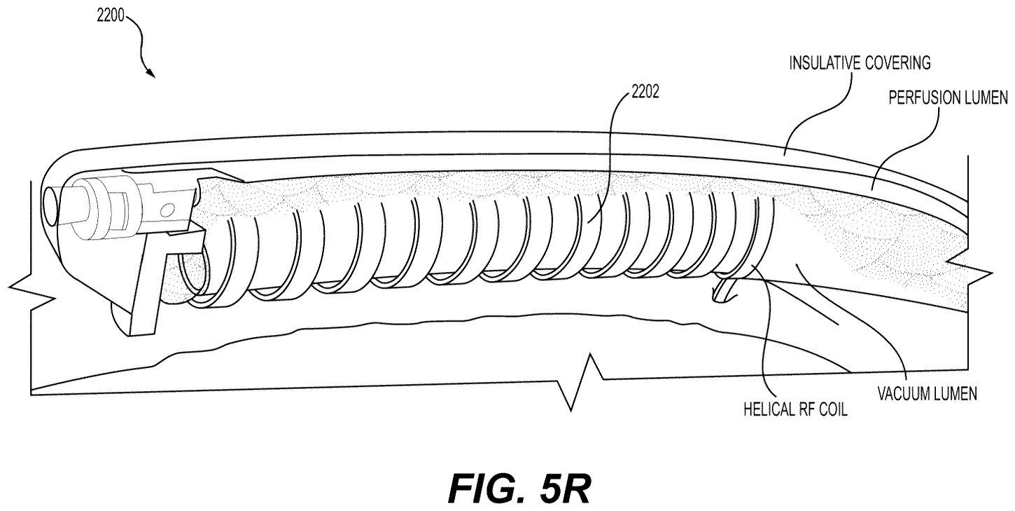

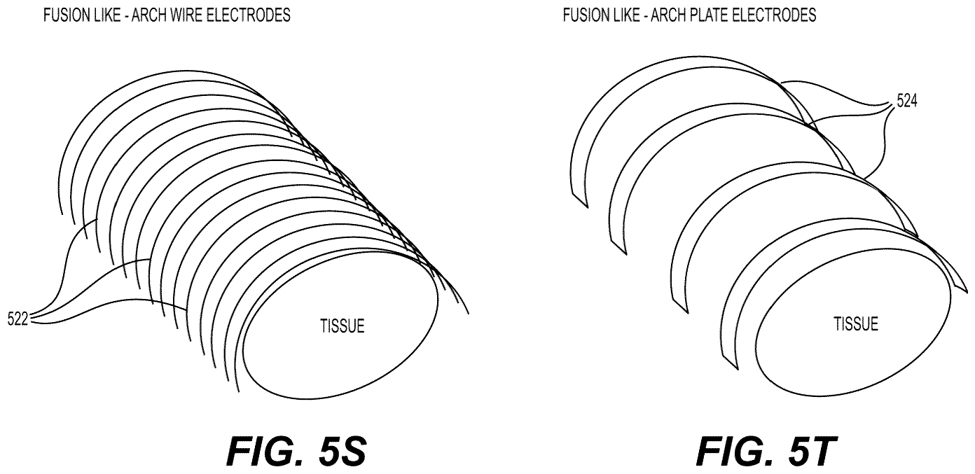

Referring to FIGS. 5A-5T, FIG. 5A illustrates an elongated, generally linear (e.g., “wire”) electrode that may be generally straight, or may include one or more curves and/or angles, and may be repeated to provide multiple electrodes as desired. FIG. 5B illustrates a point (e.g., “spot”) electrode that may be generally circular or may have other shapes, such as a sheathed electrode that exposes a point. FIG. 5C illustrates a segmented electrode that includes a plurality of discreet, generally rectangular segments, though segments with other similar or differing shapes may be utilized. In some example embodiments, the segments may be electrically coupled to one another. Such discreet portions may be arranged in a line, curve, tortuous path, stacked, or other arrangement. FIG. 5D illustrates an example nested electrode arrangement, where one or more electrodes are positioned successively inside another. In the illustrated embodiment, one or more generally annular and/or circular electrodes or electrode segments may be arranged generally concentrically within one another. Nevertheless, other enclosed shapes may be utilized such as, without limitation, triangles, rectangles, pentagons, hexagons, octagons, etc.

FIG. 5E illustrates an example squiggle electrode that includes one or a series of elongated electrodes having a plurality of opposite direction curves that may resemble a sinusoidal curve. FIG. 5F illustrates an example perpendicular electrode arrangement that includes a first segment disposed generally orthogonal to a second segment. The segments may be connected, in the form of a single electrode, or may not be connected, in the form of two separate electrodes or a segmented electrode. In the illustrated embodiment, each segment includes a generally straight electrode. FIG. 5G illustrates an example parallel electrode arrangement that includes a first segment disposed generally parallel to a second segment. In the illustrated embodiment, each segment includes a generally straight electrode. And more than two parallel electrodes may be utilized depending upon the application.

FIG. 5H illustrates an example continuous electrode, while FIG. 5I illustrates two example electrode arrays. Generally, a continuous electrode may have a continuous surface that is presented to a tissue, regardless of the shape of the electrode. Generally, a discontinuous electrode array may include two or more segments having separated tissue contacting surfaces. Each segment may have any shape, such as generally circular and/or generally straight. In some example embodiments, two or more segments of an electrode array may be electrically connected. In some example embodiments, two or more segments of an electrode array may be electrically isolated from each other, so that each delivers differing or the same electrical signals to the tissue, for example.

FIG. 5J illustrates example plate electrodes that may include a two or three dimensional electrode surface having a substantial width in view of its length, regardless of its shape. Plate electrodes may be provided in continuous or segmented configurations, for example. FIG. 5K illustrates an example electrode configuration including multiple pairs of cooperating electrodes. FIG. 5L illustrates example raised electrodes. Generally, raised electrodes may protrude from a surrounding surface of the PFA device. In some embodiments, one or more electrodes may be disposed flush with the surrounding surface. That is, the tissue contacting surface of the electrode may be substantially coplanar with the surrounding surface. In some embodiments, one or more electrodes may be recessed within the surrounding surface. That is, the tissue contacting surface of the electrode may be inset with respect to the surrounding surface. FIG. 5M illustrates an example electrode configuration including raised electrodes positioned opposite a plate electrode.

FIGS. 5N-5T are simplified section views of alternative example electrode configurations which may be used, for example, in connection with PFA devices generally similar to PFA device 200, 300, 400, 2200 according to at least some aspects of the present disclosure. It will be understood, however, the similar configurations may also be utilized in other PFA devices. Specifically, FIG. 5N depicts a longitudinal section view of an example elongated (e.g., wire) electrode configuration, including two electrodes 502, 504. In this embodiment, the elongated electrodes 502, 504 may be arranged as an opposed pair such as for bipolar operation. The electrodes 502, 504 may be oriented generally longitudinally and/or may be at least partially recessed within the stabilizer 312. FIG. 5O depicts a longitudinal section view of an example multiple elongated (e.g., wire) electrode configuration, including four electrodes 506, 508, 510, 512. In this embodiment, the elongated electrodes 506, 508, 510, 512 may be arranged as two opposed pairs and/or may be oriented generally longitudinally. But greater than two pairs of electrodes is within the scope of the instant disclosure. FIG. 5P depicts a lateral section view of a continuous electrode configuration 514 and a segmented electrode configuration 516. In the illustrated embodiment, individual electrodes 516A, 516B, 516C of the segmented electrode configuration 516 may be electrically connected as a group or individually driven, but may individually contact the target tissue.

FIG. 5Q depicts a lateral view of an example electrode arrangement comprising opposed, tissue penetrating needle electrodes. In the illustrated embodiment, a first jaw 517 includes at least one needle electrode 518 extending therefrom. A second, opposed jaw 519 includes at least one needle electrode 520 extending therefrom. In the illustrated embodiment, the needle electrodes 518, 520 are disposed in respective arrays. The spacing between the needles 518, 520 may be fixed at a known, specified distance. While the illustrated embodiment includes jaws 517, 519 that may represent jaws 210, 212 of a clamp-type PFA device (e.g., PFA device 200 depicted in FIG. 2), it will be understood that such an opposed needle arrangement may be utilized with other configurations of PFA devices, such as PFA devices 300, 400.

FIG. 5R depicts a partial cutaway perspective view of the minimally invasive PFA device 2200 illustrated in FIG. 22, where the electrode may be generally in the form of a helical electrode 2202. FIG. 5S depicts a simplified distal perspective view of an example electrode configuration comprising a plurality of arch wire electrodes 522. In this embodiment, the arch wire electrodes 522 may be disposed at least partially within a stabilizer (FIG. 3A) and may be generally parallel, laterally oriented, and/or configured to engage the target tissue on respective concave surfaces. And FIG. 5T depicts a simplified distal perspective view of an example electrode configuration comprising a plurality of arch plate electrodes 524. In this embodiment, the arch plate electrodes 524 may be disposed at least partially within a stabilizer (FIG. 3A) and may be generally parallel, laterally oriented, and/or configured to engage the target tissue on respective concave surfaces. Generally, the arch plate electrodes 524 of FIG. 5T may be similar to the arch wire electrodes 522 of FIG. 5S; however, in some embodiments, the arch plate electrodes 524 may be wider (e.g., in a longitudinal direction) than the arch wire electrodes 522.

In some example embodiments, clamp-type PFA devices (e.g., similar to PFA device 200) may include various features. Generally, clamp-type devices may be configured for dynamic closure and/or static closure.

Example embodiments configured for dynamic closure may utilize static jaws and/or dynamic jaws. For example, a static jaw (e.g., a jaw which does not change orientation during use) may be dynamically configured by use of a spring closure mechanism. In some such embodiments, the closure force is substantially provided by the spring force, and the jaw separation in the closed configuration depends on the tissue thickness and compressibility. In other embodiments, a static jaw may be dynamically configured by utilizing a user-applied closing force. Thus, the jaw separation and closure force in the closed configuration are directly controlled by the user. Example embodiments including dynamic jaws may include a compressible jaw surface, conformable jaws (e.g., jaws that deform when subject to design closure forces), and/or flexible jaws.

Some example embodiments configured for static closure may utilize a pressure set. That is, a closure force may be applied up to a pre-set, desired level. In such a circumstance, a further applied closure force would be ineffective to further close the jaws.

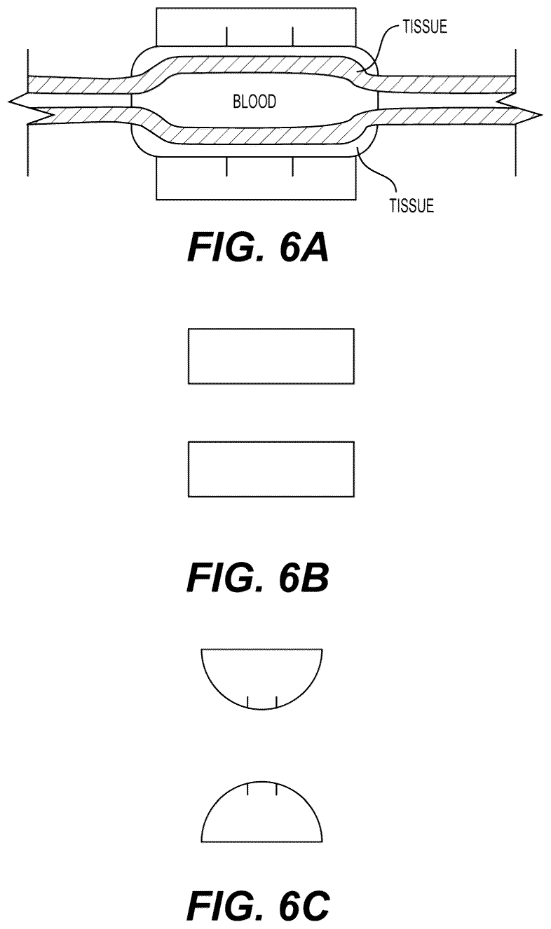

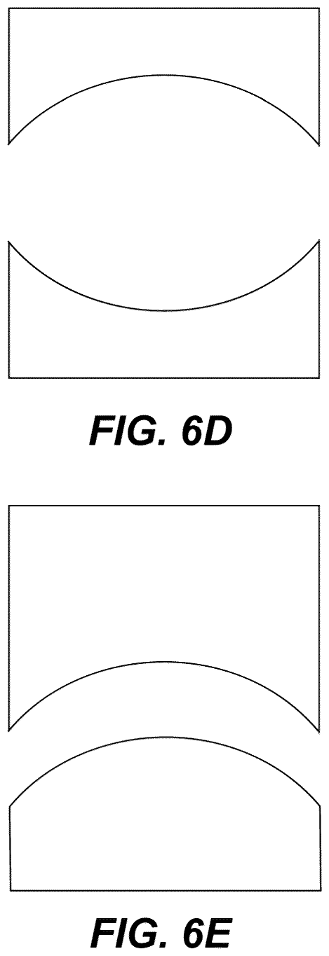

Referencing FIG. 6A, some example embodiments configured for static closure may utilize a fixed distance set. That is, the jaws are closed to a pre-determined jaw spacing, regardless of the closing force necessary to achieve such spacing. In some example embodiments employing a fixed distance set, a PFA device may utilize both clamping and vacuum tissue engagement features. For example, FIG. 6A depicts a simplified section view of a vacuum-clamp configuration, according to at least some aspects of the present disclosure. In the illustrated embodiment, the jaws are positioned around the target tissue and are moved to the closed configuration. Vacuum is applied to the jaws to maintain or increase desired tissue contact with the jaws.

Some example embodiments may be configured for hybrid set distance/dynamic closure operation. For example, an initial closure of a clamp may be performed to a set distance. This may facilitate a PFA, such as at a fixed or known V/cm. Then, the clamp may be closed dynamically, such as in preparation for RF ablation. Some example embodiments may include closure mechanisms that provide such sequences of operations, or that may be switchable (e.g., user-selectable) between such modes of operation. In some example embodiments, a closure mechanism mode of operation (e.g., dynamic vs. fixed distance) may be determined in connection with selecting an output of an electrosurgical generator (e.g., PFA vs. RF ablation). In alternative embodiments, dynamic closure may be performed first, followed by set distance closure, such as to perform RF ablation followed by PFA.

Some example embodiments may utilize a variable distance set. That is, the jaws may be closed to a particular distance, such as may be decided by a user and/or indicated by detents or a visible scale, but that distance may vary from ablation to ablation.

Referring to FIGS. 6B-6E, some example embodiments may utilize opposed jaws including cooperating tissue engagement features. For example, insulator portions of jaws proximate the electrodes may be configured with various shapes for engaging the target tissue. FIG. 6B illustrates a baseline example embodiment including substantially flat opposed tissue engagement surfaces; FIG. 6C illustrates an example embodiment including opposed convex tissue engagement surfaces; FIG. 6D illustrates an example embodiment including opposed concave tissue engagement surfaces; FIG. 6E illustrates an example embodiment including a convex tissue engagement surface opposing a cooperating concave tissue engagement surface; all according to at least some aspects of the present disclosure.

In some example embodiments, the tissue engagement surfaces (e.g., insulators) may be substantially rigid. That is, the insulators do not substantially deform under design loads. In some example embodiments, the tissue engagement surfaces (e.g., insulators) may be substantially compliant. That is, the insulators may be configured to deform, such as to conform to the target tissue, when subjected to design loads. In some example embodiments, the tissue engagement surfaces may be partially rigid and/or partially compliant, as may be suitable to achieve desired tissue contact.

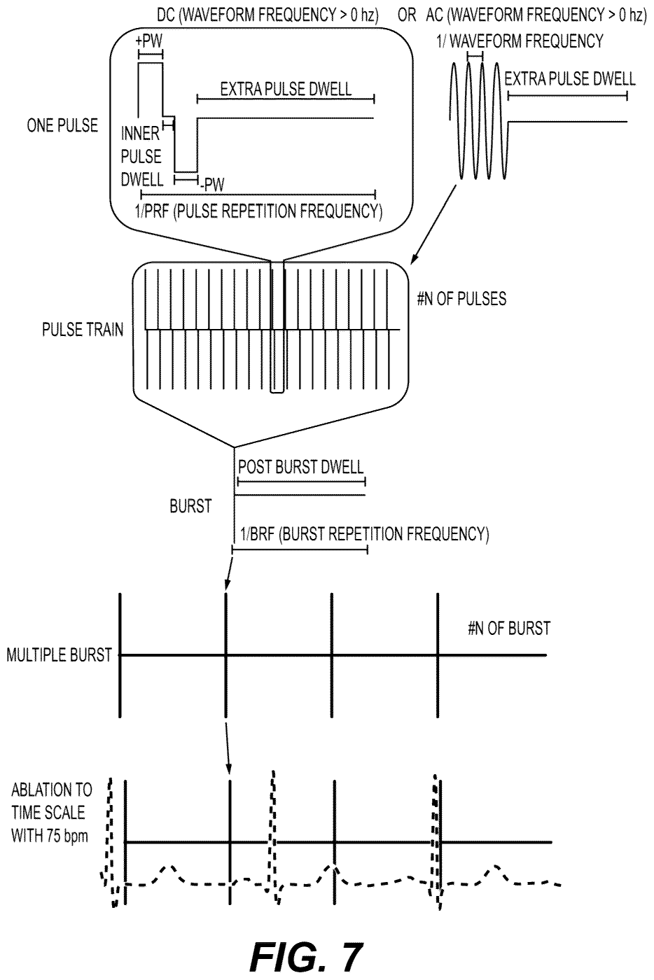

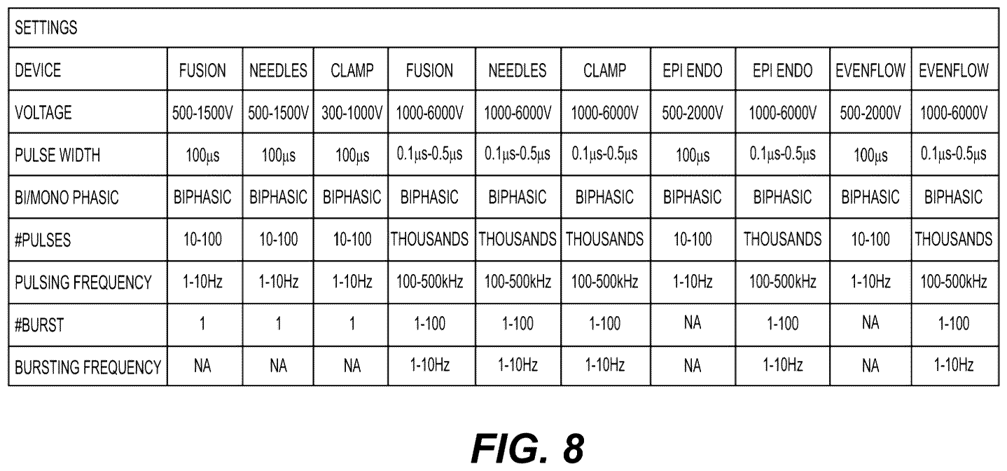

Turning to FIG. 7, a graphical illustration of the composition of an example multiple burst PFA signal is depicted, according to at least some aspects of the present disclosure. And FIG. 8 depicts a table listing example PFA signal parameters that may be used in connection with a variety of PFA devices, according to at least some aspects of the present disclosure. In FIG. 8, “fusion” refers to a device similar to PFA device 300 shown in FIG. 3, “needles” refers to a device similar to PFA device 400 shown in FIGS. 4A-4C, “clamp” refers to a device similar to PFA device 200 shown in FIG. 2, “epi endo” refers to a PFA system comprising an epicardially positioned PFA device operated in cooperation with an endocardially positioned PFA device, and “evenflow” refers to a PFA device comprising a plurality of electrodes operated at predetermined, different voltages. The listed parameters are merely examples and should not be considered limiting in any way.

In various example embodiments, PFA signals may comprise monophasic pulses and/or biphasic pulses. Individual pulses may comprise square waves and/or voltage may vary over time. For example, an individual pulse may comprise a generally sinusoidal waveform. Individual pulses may be delivered in a burst (e.g., a pulse train). A series of multiple bursts may be delivered. Some example embodiments may deliver pulses at particular predetermined times relative to a patient's heartbeat.

Pulse characteristics may be varied and selected to achieve a desired result. For example, alternating current (“AC”) or direct current (“DC”) waveform, pulse amplitude, number of pulses in a pulse train, number of bursts, pulse repetition frequency, burst repetition frequency, pulse width (e.g., nanosecond or greater), etc., may be varied. In some example embodiments, some or all of the characteristics may remain substantially constant. In some example embodiments, one or more characteristics may change, such as during the course of an ablation. For example, some characteristics may be programmed to change with time.

In some example embodiments, operation of a PFA system may be configured to measure one or more parameters, in real-time or time delayed, associated with an ablation operation and/or to utilize data pertaining to such parameters in connection with controlling the delivery of PFA energy. In some example embodiments, based at least in part upon detection and/or measurement of some parameters, some aspects of the PFA energy delivery may be enabled and/or inhibited. In some example embodiments, one or more aspects of a PFA energy delivery may be adjusted and/or controlled based at least in part upon detection and/or measurement of one or more parameters.

In some example embodiments, contact force, or a parameter associated with the contact force, between an end effector and a target tissue may be measured. For example, in an embodiment employing vacuum stabilization, vacuum level may be measured. In an embodiment employing magnetic attraction, the magnetic attraction force may be measured.

In some example embodiments in which the spacing between electrodes may vary, such spacing may be measured as described elsewhere herein.

In some example embodiments, one or more temperatures may be measured. For example, one or more end effector temperatures, electrode temperatures, and/or tissue temperatures may be measured.

In some example embodiments, tissue conductance may be measured. For example, tissue conductance may be measured using the same electrodes as may be used to deliver PFA energy. Alternatively, additional electrodes, different from those used to deliver PFA energy, may be used to measure tissue conductance. Tissue conductance measurements may be assessed as absolute conductance values and/or in view of a change in conductance, such as a percent conductance change resulting from an ablation. In some circumstances, tissue conductance may increase due to PFA, so such measurements may facilitate assessment of ablation effectiveness and/or progress.

In some example embodiments, current delivered in connection with PFA may be measured.

In some example embodiments, ablation time may be measured.

The present disclosure contemplates that tissue selectivity, which may refer to the ability to target destruction of specific tissues with minimal damage to other non-targeted nearby tissues, may be a relevant consideration when ablating tissues. For example, ablation of myocardial tissue may occur near the phrenic nerve, the esophagus, and coronary arteries.

The present disclosure contemplates that tissue selectivity of PFA may be influenced by various factors, including duration and intensity of the electric field, shape and size of electrodes, and electrical properties of the target tissue. Generally, PFA may be more selective for tissues with higher electrical conductivity, such as myocardium, and less selective for tissues with lower conductivity, such as fatty or fibrous tissue. In some circumstances, PFA energy delivered via electrical signals with certain characteristics may be substantially destructive to myocardium and/or may be minimally destructive to nerve tissue and/or blood vessels.

The present disclosure contemplates that, generally, electric field strength (E) (also referred to as applied electric field) is proportional to the voltage (V) applied and inversely proportional to the electrode spacing (d), as given by the following equation:

E=V/d

The electrode spacing (d) is defined as the distance between electrodes used to deliver the high-voltage electric pulses to the target tissue. In some example embodiments, IRE may be produced by electric field strengths of about 2500 V/cm to 10,000 V/cm.

The present disclosure contemplates that the electrode spacing may affect the spatial distribution of the electric field within the tissue. Specifically, as the electrode spacing increases, the electric field may become less concentrated given its distribution over a larger area, while at smaller electrode spacings, the electric field may become more focused given its concentration in specific regions of the tissue.

The present disclosure contemplates that electrode exposure may also affect the spatial distribution of the electric field within the tissue. Electrode exposure refers to the amount of electrode surface area that is in direct contact with the tissue being treated. Generally, a larger electrode exposure may result in a more uniform electric field distribution across tissue, which can lead to more effective destruction of the targeted tissue. In contrast, a smaller electrode exposure may result in a more localized electric field distribution within a smaller tissue footprint. The electrode exposure can be controlled by adjusting the size and shape of the electrode and/or by varying the distance between the electrode and the tissue. In some cases, multiple electrodes (greater than two) may be used to achieve a larger electrode exposure and/or a more uniform electric field distribution.

The present disclosure contemplates that pulse width may play an important role in determining the effectiveness of a PFA treatment. Pulse width may refer to the time during which the electric field is applied to the tissue. Generally, a longer pulse width may correspond to increased likelihood of causing IRE. However, in some circumstances, too short of a pulse width may deliver insufficient energy to the tissue to produce the desired effect.

The present disclosure contemplates that dwell may determine the amount of energy delivered to the tissue during PFA and thus may affect the extent of tissue damage. Dwell may refer to the time between individual pulses and/or the time between a packet or group of pulses.

The present disclosure contemplates that pulse repetition frequency may affect the duration and/or frequency at which the tissue is exposed to the electrical field, which may impact efficacy, selectivity, and/or safety, in some circumstances. Generally, pulse repetition frequency refers to the frequency at which electric pulses are delivered during PFA. In some circumstances, increasing the pulse repetition frequency may increase selectivity and reduce the likelihood or magnitude of undesired muscle stimulation.

The present disclosure contemplates that the number of pulses delivered to the tissue may affect the extent of tissue destruction and/or the effectiveness of the treatment. The number of pulses may refer to the total number of discrete times that high-voltage current is applied to a target tissue during a particular treatment. Generally, reducing the number of pulses for a given high-voltage current may reduce the potential for undesired heating of tissue.

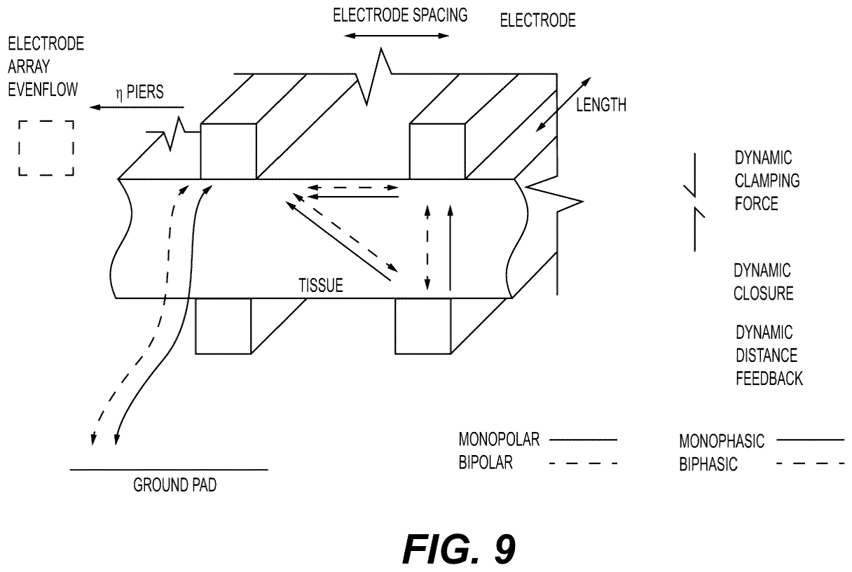

Referring to FIG. 9, the following description of bipolar/monopolar configurations and biphasic/monophasic signals according to at least some aspects of the present disclosure is explained.

The terms “bipolar” and “monopolar” may refer to the electrical configuration of the electrodes used to deliver the high-voltage electric pulses to the tissue being treated. Generally, in a monopolar configuration, a single active electrode (or group of electrodes) may be used to deliver the electric pulses to the tissue, while another electrode and/or grounding pad is typically placed elsewhere on the patient's body to complete the circuit. Such a configuration may result in a less controlled electric field distribution and/or may potentially damage healthy tissue in the vicinity of the treatment area. In a bipolar configuration, two active electrodes (or groups of electrodes) may be placed near the tissue being treated, with the high-voltage electric pulses being delivered between the two electrodes. Such a configuration may result in a more localized electric field distribution, which may reduce the risk of damage to healthy tissue outside the treatment area. Although both bipolar and monopolar configurations have been used in PFA, in some circumstances, bipolar configurations may provide some potential safety and/or effectiveness advantages. For example, in some circumstances, bipolar configurations may provide improved electric field control and/or more controlled lesion formation and/or may cause less skeletal muscle stimulation.

The terms “biphasic” and “monophasic” may refer to the waveform of electric pulses used in PFA treatment. A monophasic pulse may include a single, high-voltage electric field that is applied to the tissue for a short duration. Such a pulse may be thought of as a unidirectional wave that propagates through the tissue. A biphasic pulse may include two pulses of opposite polarity that are applied in succession. The polarity of the electric field is reversed between the two pulses, resulting in a bidirectional waveform that oscillates back and forth through the tissue. In some circumstances, biphasic pulses may be more effective in disrupting the cell membranes of some tissues, as compared to monophasic pulses.

The present disclosure contemplates that, in some circumstances, delivering electrical energy to bodily tissues may result in muscle contraction. In some circumstances, muscle tissue may contract due to direct stimulation by the electrical energy. In some circumstances, muscle tissue may contract due to stimulation of nervous tissue by the electrical energy. In the context of electrical ablation of cardiac tissue, electrical energy may cause stimulation of cardiac muscle and/or stimulation of non-cardiac, skeletal muscle. For example, such stimulation may include involuntary contractions and/or twitching.

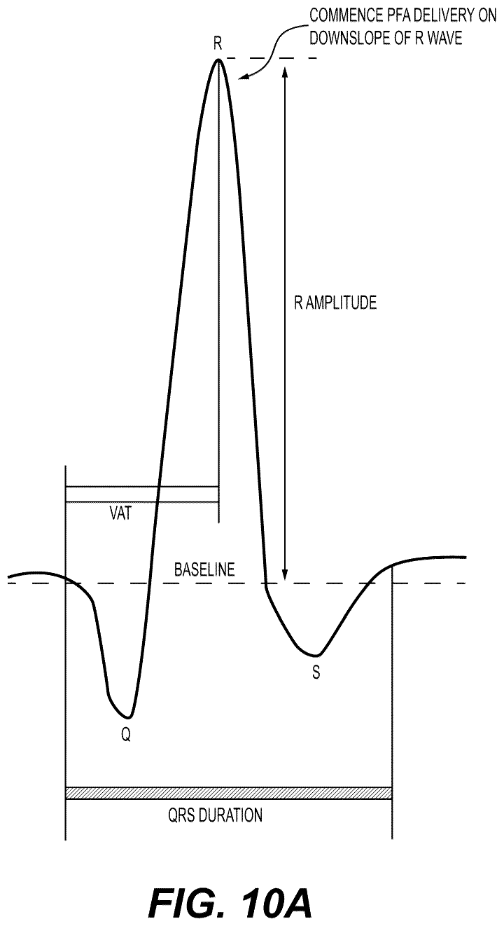

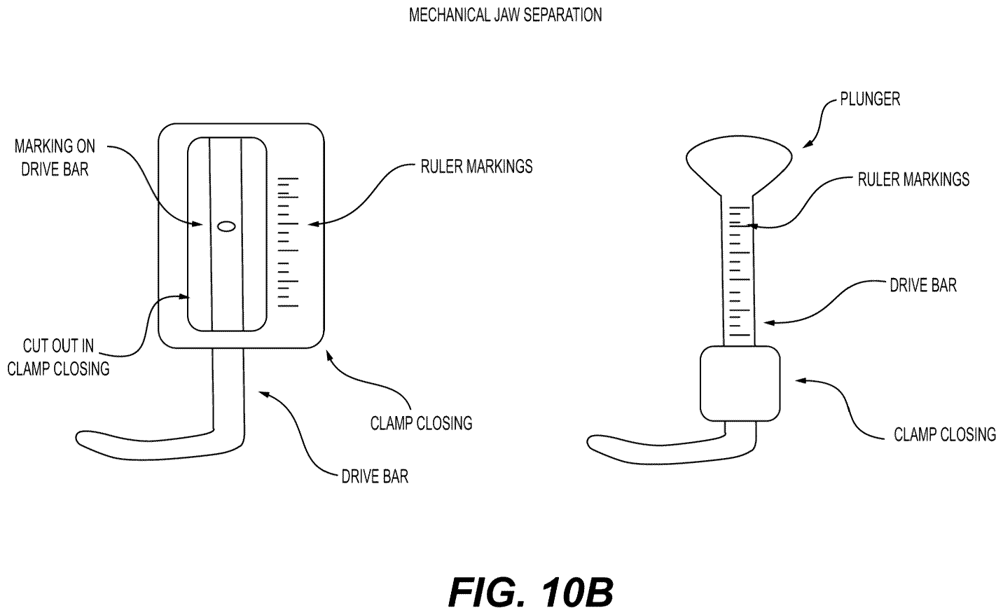

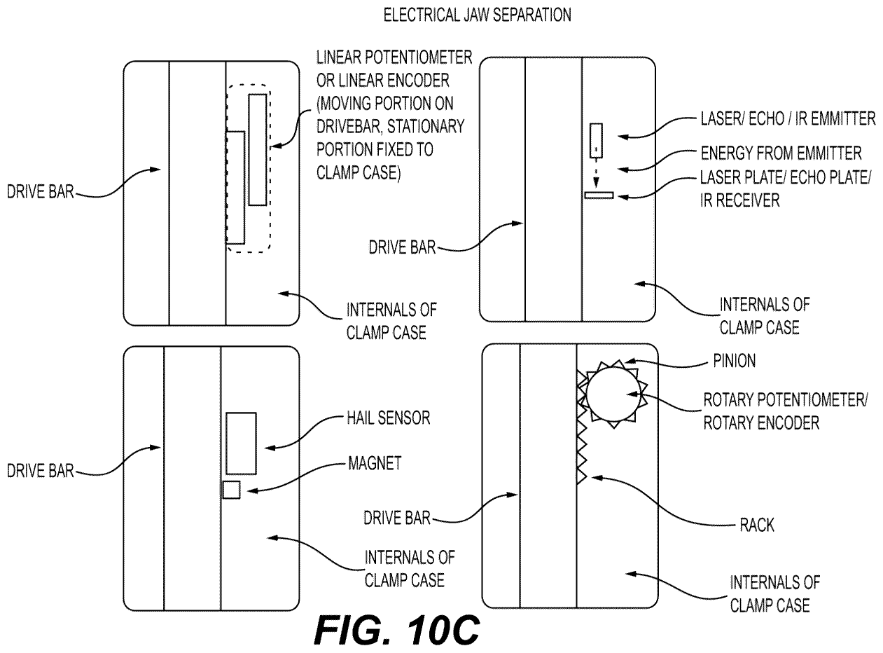

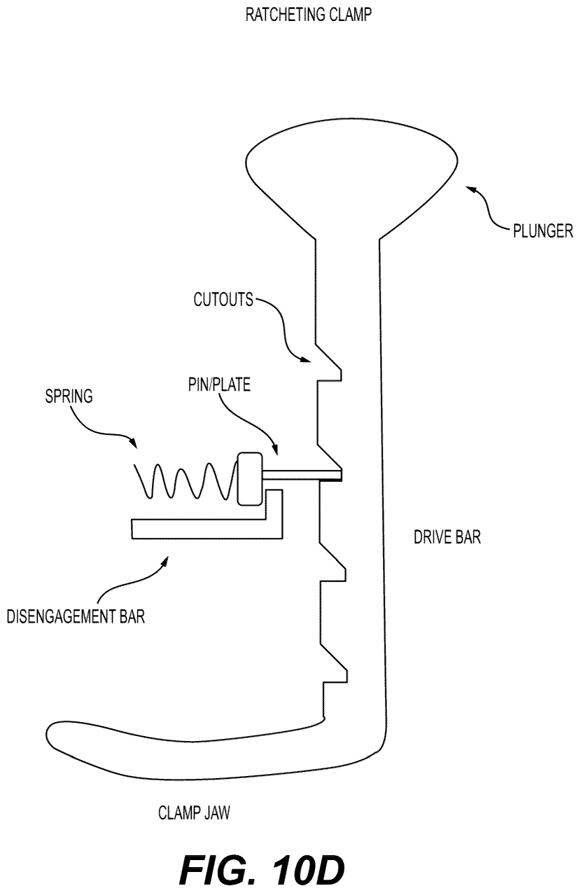

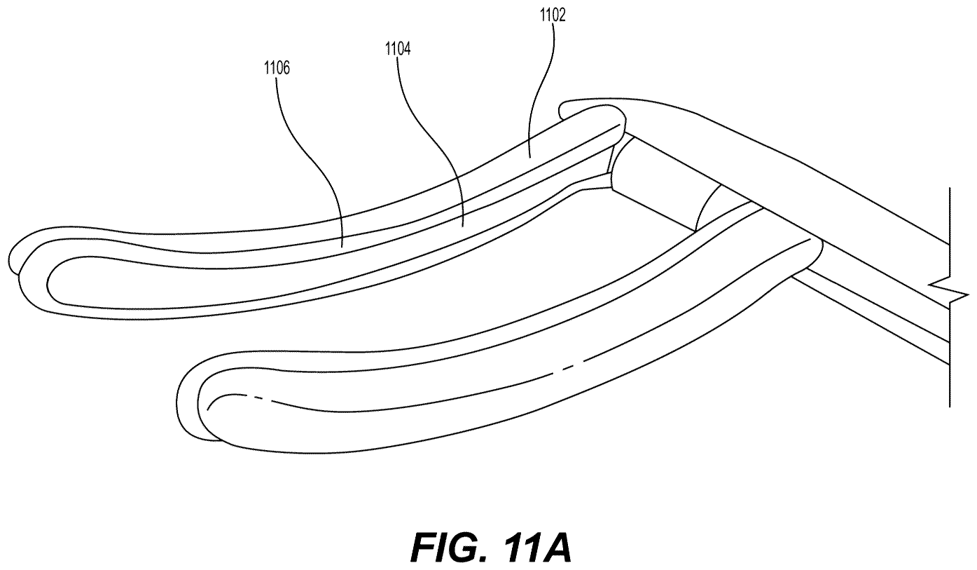

In some example embodiments, delivery of PFA energy to cardiac tissue may be timed to coordinate with the patient's heartbeat. For example, delivery of PFA energy may be timed to align with specific portions of the cardiac cycle and/or not to align with specific portions of the cardiac cycle. For example, electrical energy may be applied beginning when the heart is in its refractory period, which may reduce the likelihood of muscle spasm. For instance, as depicted in FIG. 10, a plot of an example ECG trace according to at least some aspects of the present disclosure is disclosed. In some embodiments, PFA energy delivery may commence on the downslope of the R-wave, which may reduce the likelihood of undesired, abnormal heart stimulation and/or arrythmia. Specifically, on the downslope of the R-wave, the cells have already depolarized, so they are generally unable to react to the PFA signal. In some circumstances, by applying the first of a series of pulses on the downslope of the R-wave, subsequent pulses may be applied at differing frequencies without causing adverse effects. In particular, regardless of the frequency of the subsequent pulses, the heart may be stimulated to beat at a maximum rate of about five beats per second and is likely to return to normal sinus rhythm after the ablation.

In some example embodiments employing pacing signals to drive the heart at a known rate, PFA energy delivery may be timed to coordinate with pacing signals.

In some example embodiments, PFA energy delivery parameters may be selected to reduce the likelihood of undesired cardiac and/or skeletal muscle stimulation. For example, delivering electrical energy at frequencies of about 100 kHz or greater may result in less muscle stimulation.

The present disclosure contemplates that arcing may occur between electrodes at some voltages used for PFA, leading to undesired tissue burns, cardiac arrest, hearing loss, blindness, nerve damage, and/or death depending upon the placement of PFA electrodes. In other PFA circumstances, arcing may occur between an electrode of a PFA device and the patient's tissue (e.g., target tissue or non-target tissue). Accordingly, mitigation of unintended arcing may be a consideration for the design and operation of PFA systems.

In some example embodiments, arcing may be reduced by ensuring sufficient contact force or pressure between a PFA electrode and a target tissue. For example, some embodiments may utilize vacuum stabilization to increase contact pressure. The vacuum pod may include fluid flow with conductive fluid to ensure tissue to electrode coupling. Some embodiments may utilize a clamp-type configuration to increase contact pressure. Some embodiments may utilize an expandable structure to increase contact pressure.

In some example embodiments, arcing may be reduced by covering, irrigating, and/or submerging electrodes and/or tissues in a dielectric fluid, such as deionized water.

In some example embodiments comprising a plurality of electrodes, one or more electrodes may be selectively activated and/or disabled. For example, one or more electrodes in contact with a target tissue may be activated and/or one or more electrodes not in contact with the target tissue may be disabled/deactivated in connection with a particular application of PFA energy. For example, one or more electrodes not contacting any tissue and/or one or more electrodes in contact with tissue other than the target tissue may be disabled. The electrodes may be manually disabled by the user or automated by electrical contact testing with applied voltage to confirm tissue contact or by any combination thereof. In some example embodiments, some electrodes may be selectively utilized for particular ablation modalities (e.g., PFA, RF).

In some example embodiments including PFA devices comprising two or more electrodes, a PFA device may be constructed so that the spacing between adjacent electrodes is sufficient to avoid arcing at a desired voltage. For example, given a maximum voltage differential between two adjacent electrodes, a minimum spacing may be determined that avoids arcing. In some embodiments, such spacing may be fixed when the device is constructed by rigidly disposing the electrodes at a desired spacing. In some embodiments, such as clamp-type devices with electrodes disposed on opposed jaws, mechanical controls may be incorporated that limit the closing movement of the jaws to a minimum separation distance providing sufficient electrode spacing to avoid arcing, and/or incorporation of electrical controls to inhibit operation of the electrodes if the electrode spacing is insufficient. Such controls may be disposed in the end effector, acting on or close to one or more jaws, and/or in a handle, acting on or close to a user-operated actuation element, and/or in the PFA unit 102 as physical circuitry and/or programming code.

In some example embodiments, potential arcing conditions may be detected and/or prevented. For example, a PFA device and/or PFA unit may be configured to prevent delivery of PFA energy when potential arcing conditions are detected. Alternatively, a PFA device and/or PFA unit may be configured to adjust its operation based upon detection of potential arcing conditions. For example, electrodes not in contact with tissue may be disabled, thus allowing application of PFA energy only through electrodes in substantial contact with tissue. In some example embodiments, parameters associated with arcing conditions may be detected and energy delivery may be terminated. For example, if voltage, current, conductivity, impedance, or other electrical parameters associated with arc initiation are detected, an example PFA unit 102 may terminate energy delivery to the PFA device.

Some example embodiments allowing variable electrode spacing (e.g., at least one electrode that is repositionable relative to at least one other electrode) may be configured to measure the electrode spacing, such as before PFA energy is delivered to the electrodes. In some clamp-type embodiments, determining the jaw separation distance may correlate with the electrode spacing. For example, if it is determined that the electrode spacing is insufficient to prevent arcing at a desired voltage, the PFA system may prevent delivery of PFA energy to the electrodes. In some example embodiments, a maximum voltage delivered to the electrodes may be adjusted based at least in part upon a detected or determined electrode spacing. That is, for example, the maximum voltage may be lower when a closer electrode spacing is detected or determined and/or the maximum voltage may be higher when a farther electrode spacing is detected or determined.

In some clamp-type PFA devices, jaw separation may be controlled and/or determined mechanically and/or electrically. Example mechanical configurations may include mechanisms configured to measure the distance between the opposed jaws, a ratcheting mechanism associated with the distance between the jaws, a window cut-out on the shaft or handle indicating the separation between the jaws, and/or ruler marks on the shaft, for example. Example electrical configurations may include magnet and Hall sensor devices, linear potentiometers, laser, echo, infrared, and/or tissue impedance.

Turning to FIG. 10B, two example embodiments configured to mechanically measure the distance between opposed jaws are illustrated. As illustrated, in some embodiments, ruler markings may be provided on a component that is stationary relative to a moving drive bar, or may be provided on a moving drive bar, for example.

Referring to FIG. 10C, four example embodiments configured to electrically and/or electronically measure the distance between opposed jaws are illustrated. As illustrated, some embodiments may include linear potentiometers (or linear encoders), Hall sensors, laser/echo/or IR distance measurement, and/or rotary potentiometers (or encoders), for example.

The present disclosure contemplates that voltages associated with PFA may be substantially greater than voltages associated with RF ablation. Accordingly, PFA devices may utilize increased electrical insulation as compared to RF-only devices. For example, it may be advantageous to construct PFA devices from nonconductive materials and/or to insulate (or increase insulation on) conductive elements of PFA devices. As an example, it may be advantageous to electrically insulate a tubular metal shaft extending between a handle and an end effector.

In some example embodiments, the material(s) from which electrodes are constructed may be selected to reduce the likelihood of arcing. For example, some electrodes may be constructed entirely of a single material having certain characteristics. In some example embodiments, a portion of an electrode (e.g., a main body portion) may be constructed from a first material and may be at least partially covered (e.g., plated or coated) with a second material, different than the first material. Example electrode materials include, without limitation, copper, gold, and nickel.

In some example embodiments, one or more electrodes may be formed to reduce the likelihood of arcing. For example, in some embodiments, relatively large radius curved edges may be less likely to arc than sharp corners and/or pointed projections. In some example embodiments, an array of a plurality of relatively smaller electrodes may be less likely to arc than a single, relatively larger electrode.

Turning to FIG. 10D, various clamp-type PFA devices according to at least some aspects of the present disclosure may utilize a ratcheting clamp mechanism. One such example of a ratcheting clamp mechanism is depicted. In the illustrated embodiment, the mechanism, specifically the interaction between the cutouts and the pin/plate, may be configured to allow the plunger, drive bar, and clamp jaw to move in a closing direction, while preventing movement in an opening direction. The disengagement bar may be used to withdraw the pin/plate from the cutouts, thus allowing the clamp jaw to be moved in the opening direction, when desired.

In some example embodiments, a PFA device may include one or more insulator and/or electrode arrangements configured to reduce the likelihood of arcing. Although the following example features are illustrated and described individually in the context of a clamp-type PFA device 200, one or more similar features may be utilized in connection with any PFA device configuration, including those generally similar to minimally invasive PFA device 300.

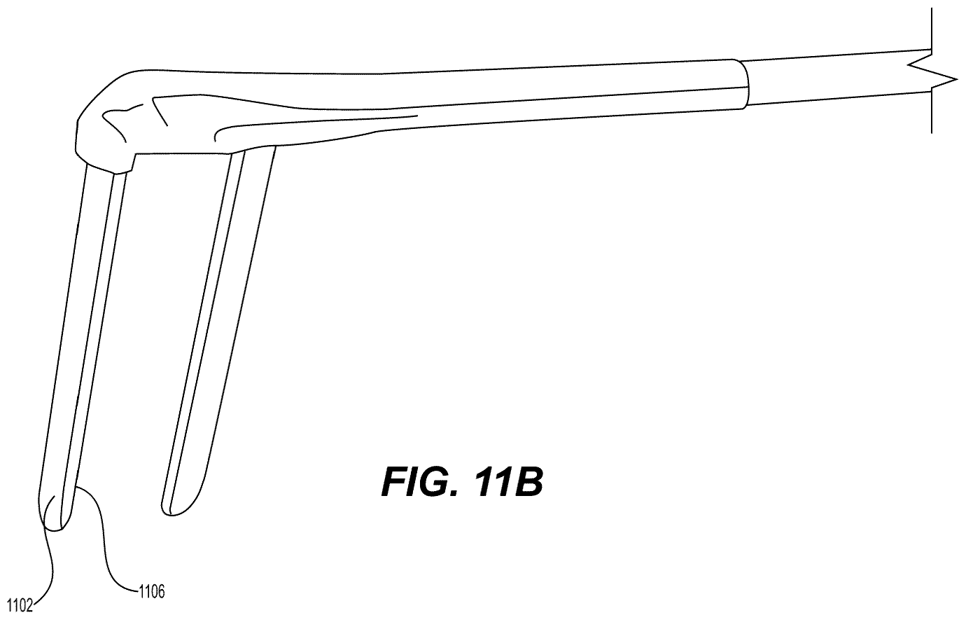

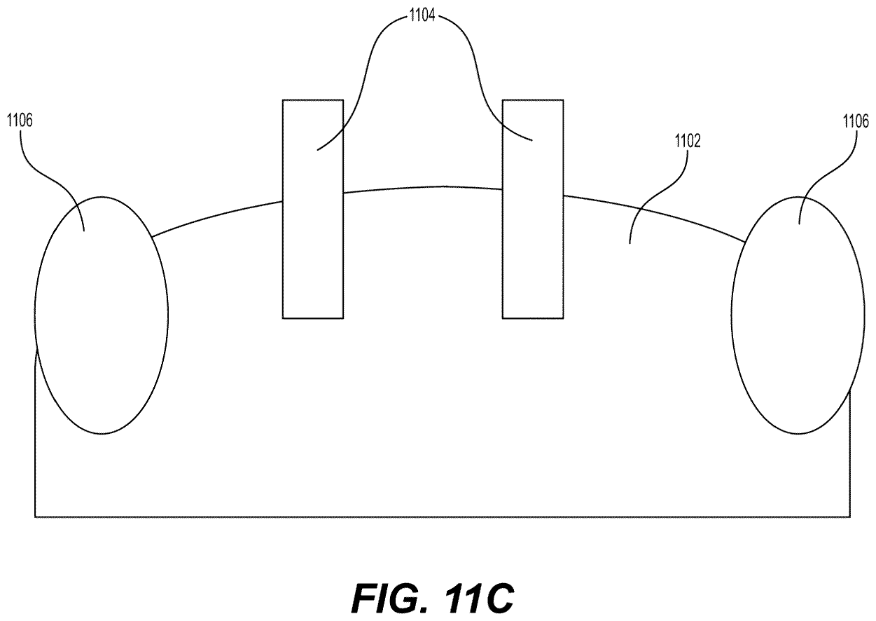

Referring to FIGS. 11A and 11B, an example insulator configuration comprising a compressible insulator at least partially circumscribing one or more electrodes is depicted. FIG. 11C is a simplified section view of the embodiment of FIGS. 11A and 11B. In the illustrated embodiment, a jaw 1102 may include one or more electrodes 1104, configured to deliver PFA energy, disposed on a tissue-engaging surface. One or more compressible insulators 1106 may at least partially circumscribe the electrodes 1104. In the illustrated embodiment, the compressible insulator 1106 may be formed from flexible, compressible, insulative tubing affixed to the perimeter of the jaw 1102. When an object (e.g., tissue) is clamped between the jaws 1102, the compressible insulators 1106 are deformed (see FIG. 11C) so that the electrodes 1104 effectively protrude from the jaw substrate and make direct contact with the object clamped. In contrast, when an object is not clamped between the jaws 1102, the compressible insulators 1106 protrude from the jaw substrate and extend outward beyond the reach of the electrodes 1104 to mitigate accidental electrical discharge to unintended objects. Insulators 1106 may be set to a height to ensure that the electrodes 1104 are never in direct contact, or to ensure a minimum electrode separation is maintained. The durometer of 1106 may be selected to achieve a specific rate of compression, that could be relatively stiffer or softer than the tissue clamped between the opposing jaws.

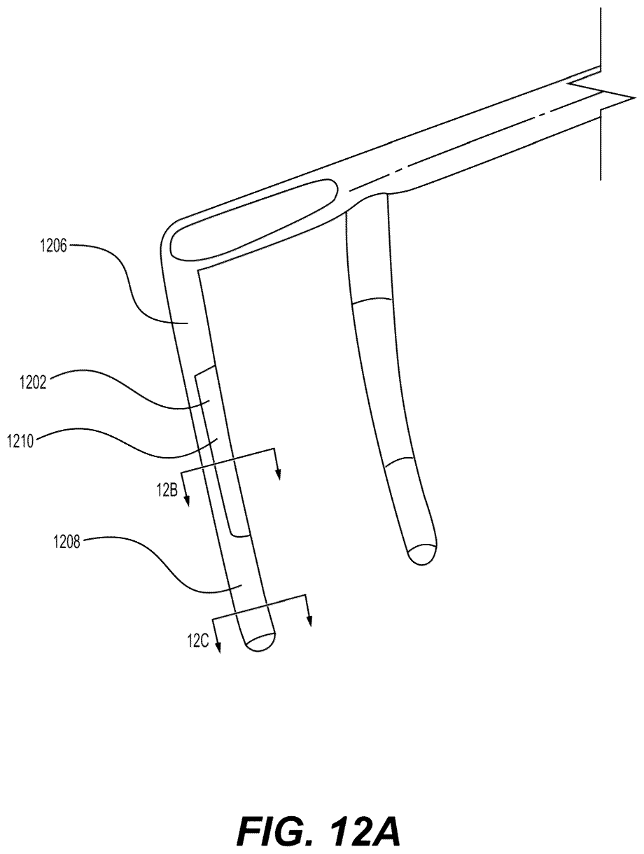

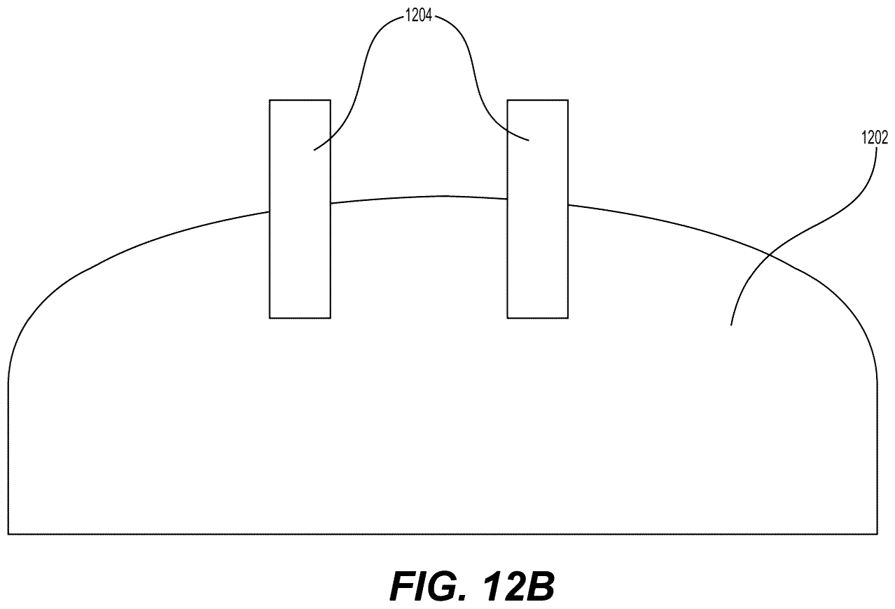

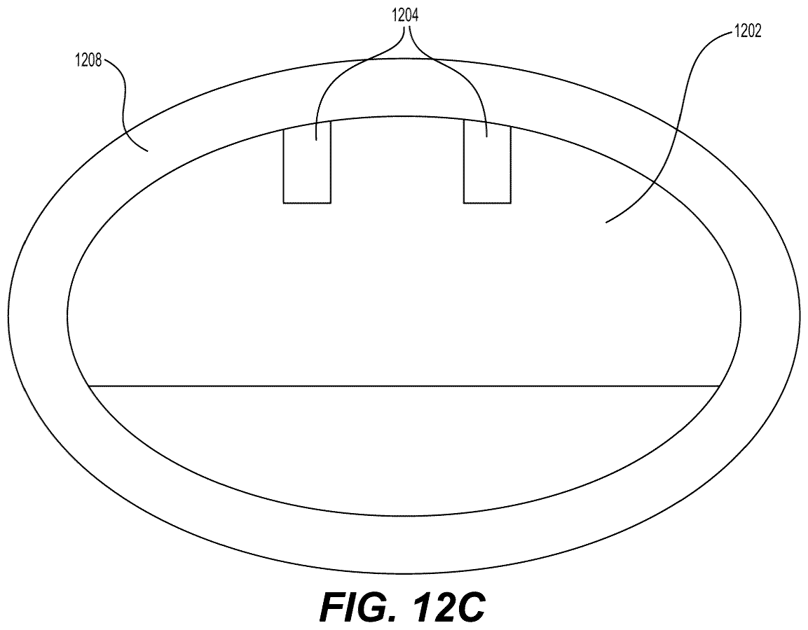

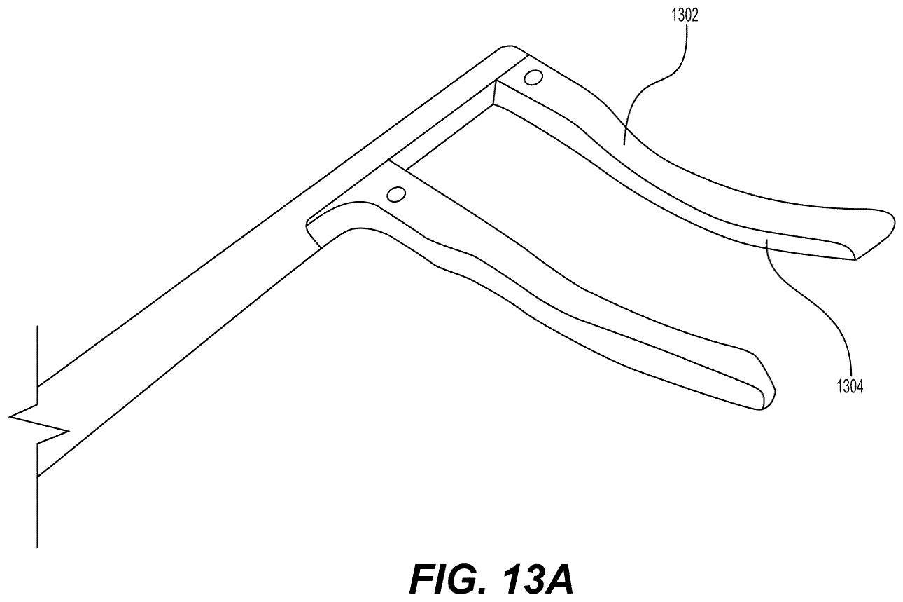

Turning to FIG. 12A, an example insulator configuration forming a shortened electrode exposure region is depicted. FIGS. 12B and 12C are simplified section views of the embodiment of FIG. 12A. In the illustrated embodiment, a jaw 1202 may include one or more electrodes 1204 configured to deliver PFA energy disposed on a tissue-contacting surface. One or more portions of the electrodes 1204 may be at least partially covered by a fixed or adjustable (sliding) insulator so that the tissue-contacting length of the electrodes is reduced. In the illustrated embodiment, a first insulator 1206 may cover a portion of the jaw 1202 proximate the heel and/or a second insulator 1208 may cover a portion of the jaw 1202 proximate the toe. Accordingly, a generally central portion 1210 of the jaw 1202 may remain uncovered, thus allowing contact between the electrodes and the target tissue. In the illustrated embodiment, the first and second insulators 1206, 1208 may be formed from silicone tape wrapped around the jaw 1202. It should be noted that insulators other than silicone may be utilized to inhibit direct contact between a portion of the electrodes 1204 and tissue.

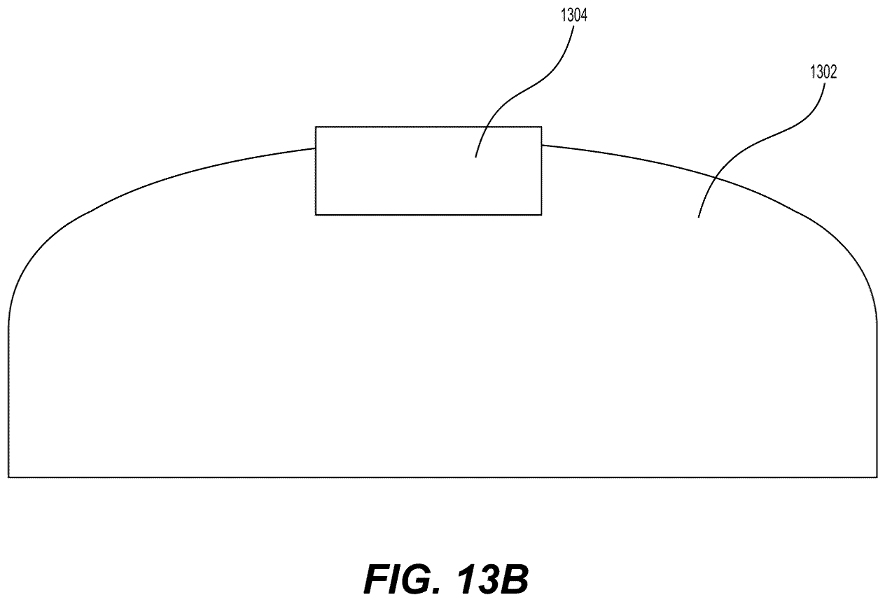

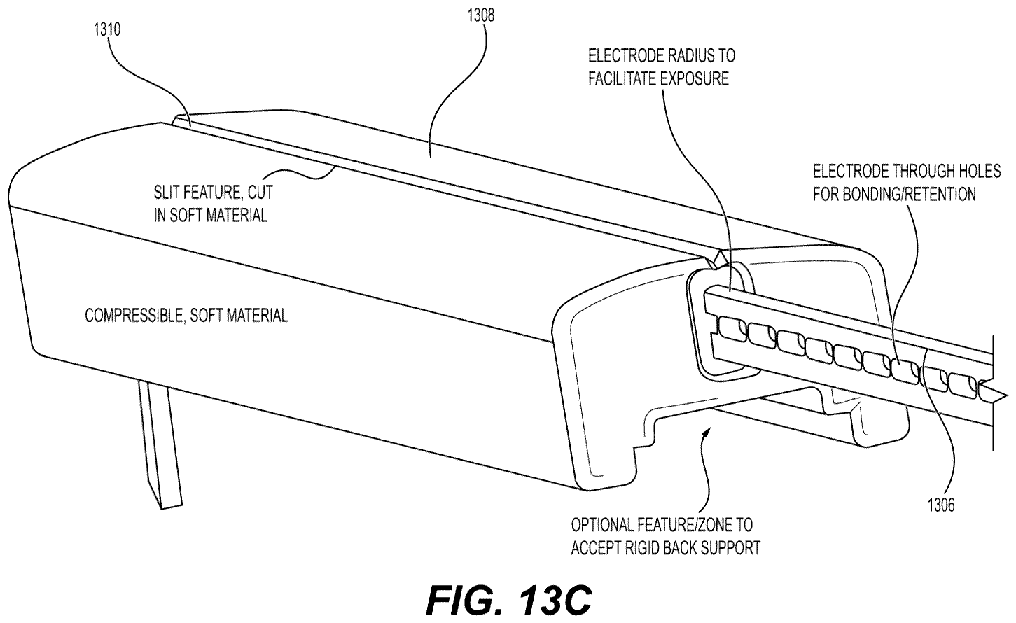

Referring to FIG. 13A, an example configuration comprising an insulated jaw is depicted. FIG. 13B is a section view of the embodiment of FIG. 13A. In the illustrated embodiment, a jaw 1302 may be constructed from non-conductive (e.g., insulative) material(s). An electrode 1304 may be embedded therein and/or disposed thereon. Thus, in contrast to embodiments in which a jaw includes externally exposed conductive material(s), the risk of arcing between an electrode and the jaw may be reduced.

Some example embodiments according to at least some aspects of the present disclosure may include one or selectively exposed electrodes. For example, some embodiments may include one or more electrodes that may be at least partially covered by one or more relatively soft, deformable insulators. In some example embodiments, one or more insulators may be configured to elastically deform and/or move to at least partially expose one or more electrodes to allow contact between the electrodes and a target tissue. Generally, in some embodiments, the electrodes may remain at least partially covered by the insulators when not in contact with the target tissue. In some example embodiments, insulators may be constructed from soft, compressible materials. The material properties may be selected so that the material moves (e.g., at least partially exposing an electrode) when clamped on tissue. In some alternative embodiments, insulators may be constructed from materials with self-healing properties.

Some example embodiments, may be constructed with one or more slits configured to facilitate elastic movement and/or electrode exposure. In various embodiments, electrodes may have any shape, including shapes configured to facilitate exposure. For example, some electrodes may be generally round, generally rectangular, generally tear-drop shaped, generally parabolic, etc.

In some example embodiments, insulators may be overmolded onto electrodes. For example, an insulator may be overmolded onto and/or bonded to a generally smooth, wire electrode. In some example embodiments, an electrode may include a coupling feature, such as a transverse through-opening, configured to facilitate bonding and retention between the insulator and the electrode. In some example embodiments, electrodes may be inserted into openings in insulators.

Some example embodiments may include a relatively rigid backing support provided within or proximate a relatively soft insulator. For example, a backing may be in the form of a flat plate and/or a grooved block, which may reduce electrode rolling and/or twisting. Some example embodiments may include intermittent post supports configured to support an electrode relative to an underlying, relatively rigid structure (e.g., jaw).

Referencing FIG. 13C, an alternative embodiment in which an electrode 1306 is selectively insulated is illustrated. In some example embodiments, the configuration of FIG. 13C may be utilized in place of the exposed electrode configuration of FIG. 13B. In the illustrated embodiment, an insulator 1308, which may be constructed of an insulative, deformable material, may be arranged to at least partially cover the electrode 1306. In a manner as generally as described below with reference to other selectively insulated electrodes, the insulator 1308 may be deformable to at least partially expose the electrode 1306, such as upon engagement with a target tissue. In the illustrated embodiment, the insulator 1308 may include a generally longitudinal slit feature 1310, which may facilitate selective exposure of the electrode 1306.

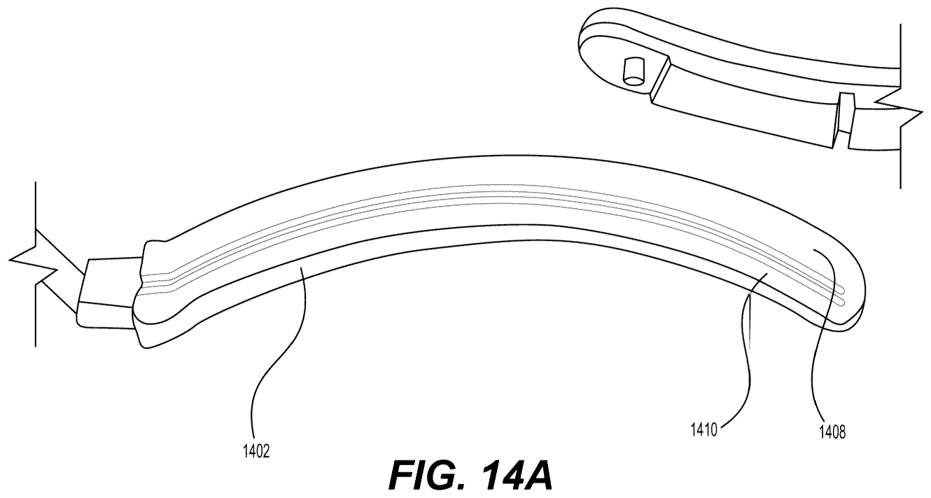

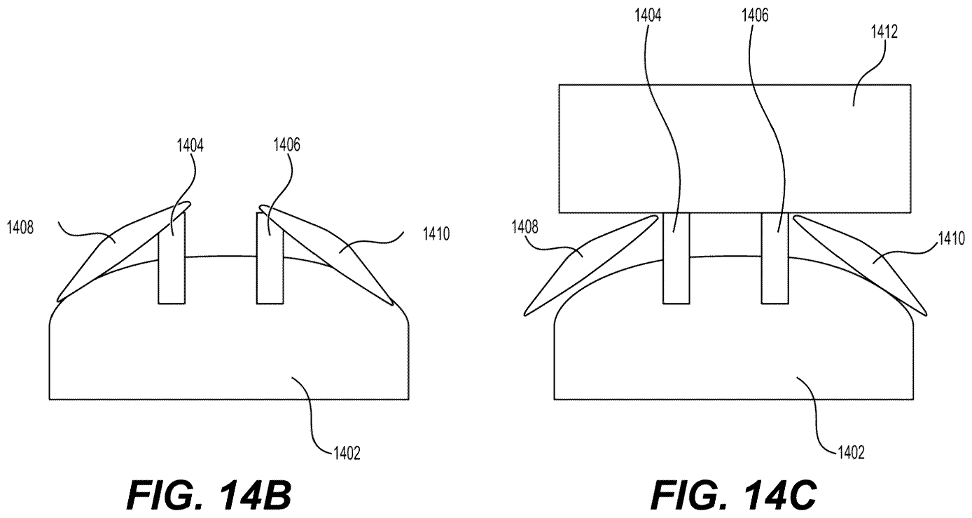

Turning to FIG. 14A, an example configuration comprising a jaw with selectively insulated electrodes is illustrated. FIGS. 14B and 14C are section views of the embodiment of FIG. 14A with the electrodes in contact with the target tissue. In the illustrated embodiment, a jaw 1402 may include one or more electrodes 1404, 1406 on a tissue-engaging surface. Referring to FIG. 14B, one or more deformable insulators 1408, 1410 may at least partially cover the one or more electrodes 1404, 1406 when the jaw 1402 is not in contact with tissue. In some example embodiments, the insulators 1408, 1410 may be constructed from flexible silicone or other suitable materials, for example. In the illustrated embodiment, each insulator 1408, 1410 substantially covers the entire length of a respective electrode 1404, 1406, with a central slit running along the length of the jaw 1402 between the inner aspects of the insulators 1408, 1410. Referring to FIG. 14C, when the jaw is placed into contact with the target tissue 1412, the target tissue 1412 may deform the insulators 1408, 1410 (e.g., generally laterally) to expose the electrodes 1404, 1406, thus allowing the target tissue 1412 to contact the electrodes 1404, 1406. If the target tissue extends substantially the full length of the jaw, substantially the full lengths of the electrodes 1404, 1406 may be exposed for contact with the target tissue 1412. If the target tissue 1412 is not in contact with the full length of the jaw 1402 only portions of the electrodes 1404, 1406 in proximity with the target tissue 1412 may be exposed. That is, portions of the electrodes 1404, 1406 not in contact with the target tissue 1412 or in close proximity to the target tissue 1412 may remain substantially covered by the insulators 1408, 1410. Accordingly, the insulators 1408, 1410 may reduce the likelihood of arcing associated with portions of the electrodes 1404, 1406 that are not in contact with the target tissue 1412.

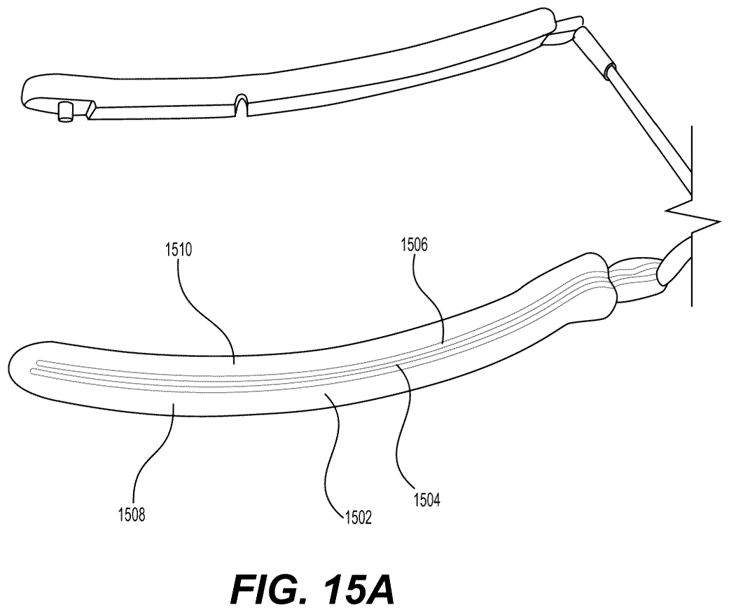

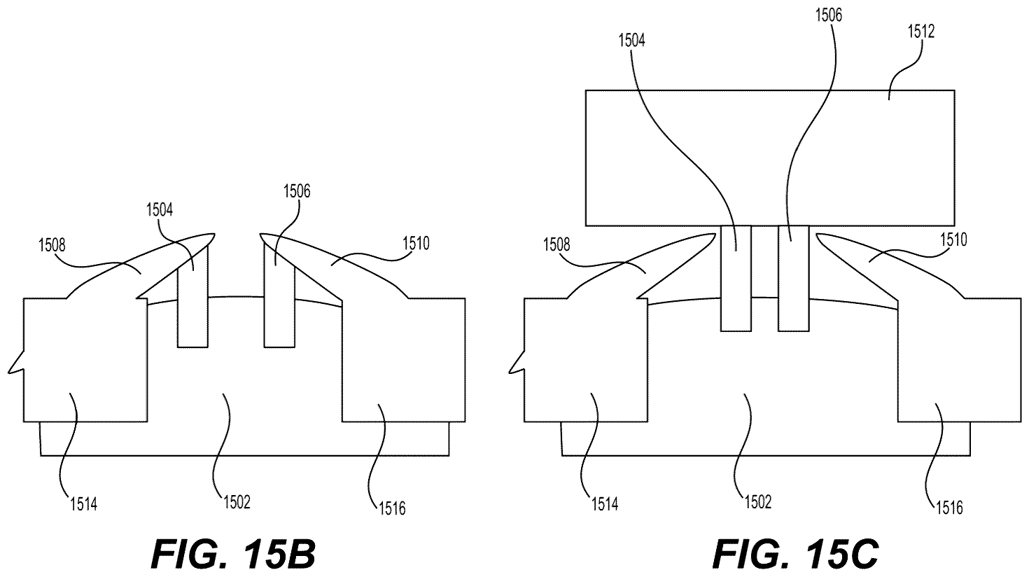

Referring to FIG. 15A, an alternative example configuration comprising a jaw with selectively insulated electrodes is illustrated. FIGS. 15B and 15C are section views of the embodiment of FIG. 15A with the electrodes in contact with the target tissue. In the illustrated embodiment, a jaw 1502 may include one or more electrodes 1504, 1506 on a tissue-engaging surface. Referring to FIG. 15B, one or more deformable insulators 1508, 1510 may at least partially cover the one or more electrodes 1504, 1506 when the jaw 1502 is not in contact with tissue. In some example embodiments, the insulators 1508, 1510 may be constructed from flexible silicone or other suitable materials. In the illustrated embodiment, each insulator 1508, 1510 substantially covers the entire length of a respective electrode 1504, 1506, with a central slit running along the length of the jaw 1502 between the inner aspects of the insulators 1508, 1510. Referring to FIG. 15C, when the jaw is placed into contact with the target tissue 1512, the target tissue 1512 may deform the insulators 1508, 1510 (e.g., generally laterally) to expose the electrodes 1504, 1506, thus allowing the target tissue 1512 to contact the electrodes 1504, 1506. If the target tissue extends substantially the full length of the jaw, substantially the full lengths of the electrodes 1504, 1506 may be exposed for contact with the target tissue 1512. If the target tissue 1512 is not in contact with the full length of the jaw 1502 only portions of the electrodes 1504, 1506 in proximity with the target tissue 1512 will be exposed. That is, portions of the electrodes 1504, 1506 not in contact with the target tissue 1512 or in close proximity to the target tissue 1512 may remain substantially covered by the insulators 1508, 1510. Accordingly, the insulators 1508, 1510 may reduce the likelihood of arcing associated with portions of the electrodes 1504, 1506 that are not in contact with the target tissue 1512. In the illustrated embodiments, the insulators 1508, 1510 of FIGS. 15A-15C differ from the insulators 1408, 1410 of FIGS. 14A-14C in that the insulators 1508, 1510 are disposed on respective, elongated base portions 1514, 1516, which may extend lengthwise along the lateral edges of the jaw 1502. In the illustrated embodiment, the insulators 1508, 1510 may be formed integrally with the respective base portions 1514, 1516. In some example embodiments, a main body of the jaw 1502 may be constructed from relatively rigid materials and/or the base portions 1514, 1516 and/or insulators 1508, 1510 may be constructed from relatively flexible, deformable materials.

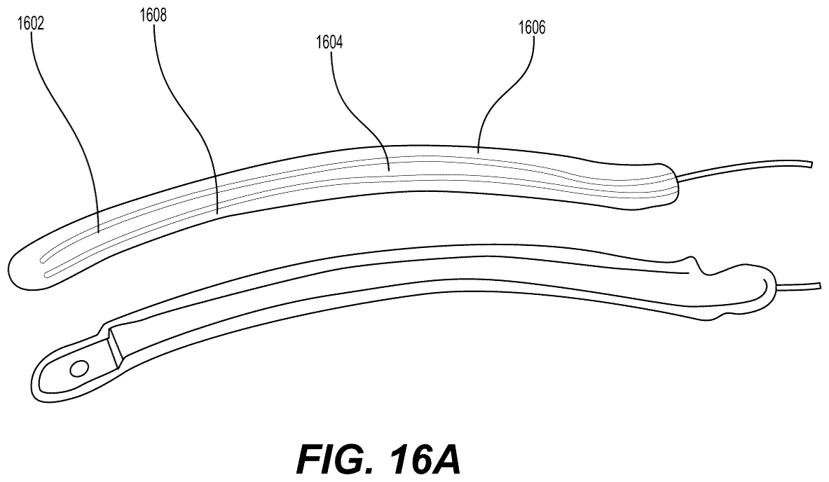

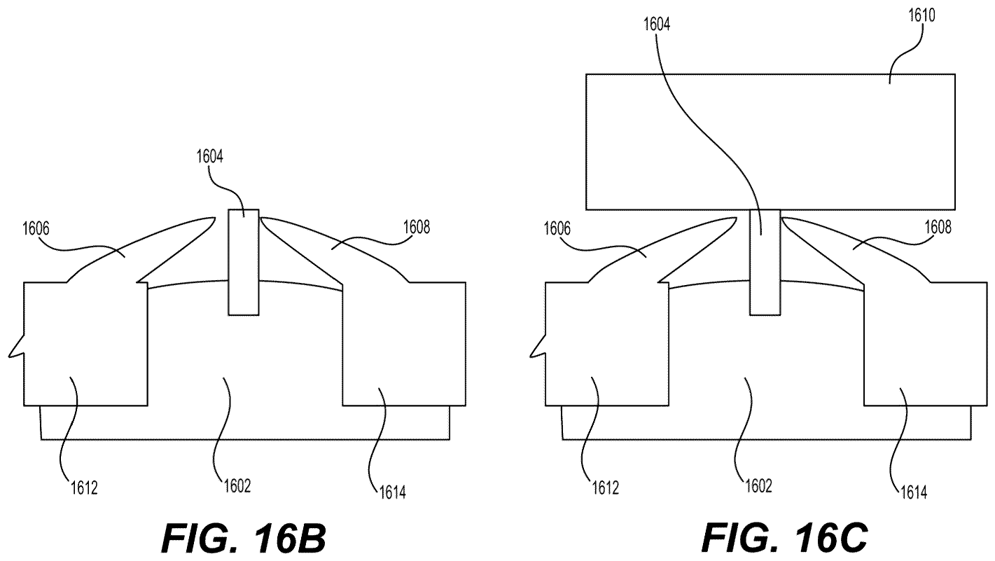

Referring to FIG. 16A, an alternative example configuration comprising a jaw with selectively insulated electrodes is illustrated. FIGS. 16B and 16C are section views of the embodiment of FIG. 16A with the electrode(s) in contact with the target tissue. In the illustrated embodiment, a jaw 1602 may include one or more electrodes 1604 on a tissue-engaging surface. Referring to FIG. 16B, one or more deformable insulators 1606, 1608 may at least partially cover the one or more electrodes 1604 when the jaw 1602 is not in contact with tissue. In some example embodiments, the insulators 1606, 1608 may be constructed from flexible silicone or other suitable materials, for example. In the illustrated embodiment, each insulator 1606, 1608 substantially covers the entire length of the electrode 1604 with a central slit running along the length of the jaw 1602 between the inner aspects of the insulators 1606, 1608. Referring to FIG. 16C, when the jaw is placed into contact with the target tissue 1610, the target tissue 1610 may deform the insulators 1606, 1608 (e.g., generally laterally) to expose the electrode 1604, thus allowing the target tissue 1610 to contact the electrode 1604. If the target tissue extends substantially the full length of the jaw, substantially the full length of the electrode 1604 may be exposed for contact with the target tissue 1610. If the target tissue 1610 is not in contact with the full length of the jaw 1602, only portions of the electrode 1604 in proximity with the target tissue 1610 may be exposed. That is, portions of the electrode 1604 not in contact with the target tissue 1610 or in close proximity to the target tissue 1610 may remain substantially covered by the insulators 1606, 1608. Accordingly, the insulators 1606, 1608 may reduce the likelihood of arcing associated with portions of the electrode 1604 that are not in contact with the target tissue 1610. In some example embodiments, the insulators 1606, 1608 may be disposed on respective, elongated base portions 1612, 1614, which may extend lengthwise along the lateral edges of the jaw 1602. In the illustrated embodiment, the insulators 1606, 1608 may be formed integrally with the respective base portions 1612, 1614. In some example embodiments, a main body of the jaw 1602 may be constructed from relatively rigid materials and/or the base portions 1612, 1614 and/or insulators 1606, 1608 may be constructed from relatively flexible, deformable materials. In the illustrated embodiments, the configuration of FIGS. 16A-16C may differ from the configuration of FIGS. 15A-15C in that it may include one elongated electrode 1604, rather than a pair of generally parallel, elongated electrodes 1504, 1506.

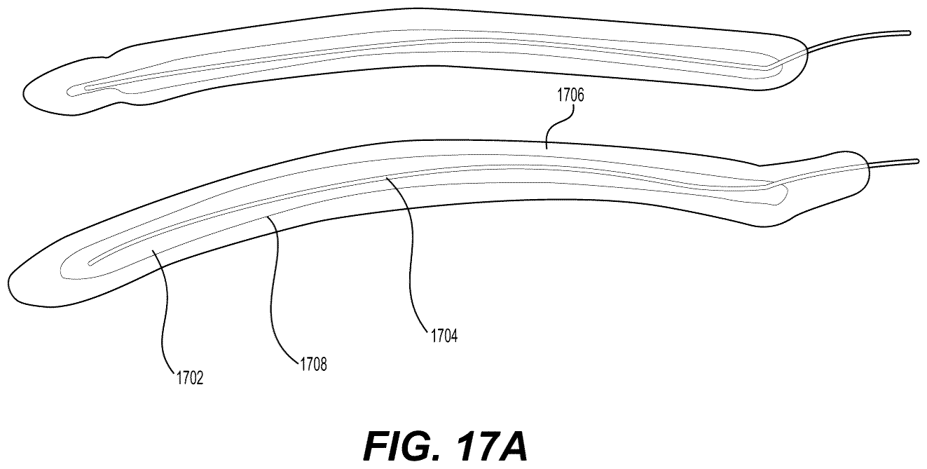

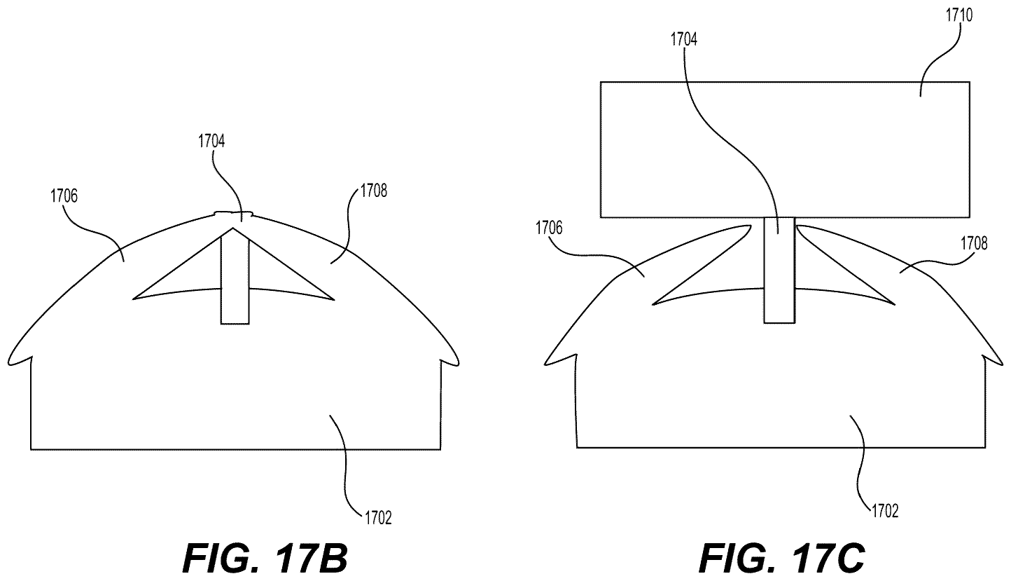

Referencing FIG. 17A, an alternative example configuration comprising a jaw with selectively insulated electrodes is illustrated. FIGS. 17B and 17C are section views of the embodiment of FIG. 17A with the electrodes in contact with the target tissue. In the illustrated embodiment, a jaw 1702 may include one or more electrodes 1704 on a tissue-engaging surface and one or more deformable insulators 1706, 1708, generally similar to the corresponding components described with reference to FIGS. 16A-16C. In the embodiment illustrated in FIGS. 17A-17C, a main body of the jaw 1702 may be formed integrally with the insulators 1706, 1708. In some embodiments, the main body of the jaw 1702 may be formed of the same material as the insulators 1706, 1708. Thus, in some embodiments, the main body of the jaw 1702 may be deformable. In some example embodiments, the main body of the jaw 1702 may be mounted to a rigid or more rigid jaw backer when assembled into a clamp-type configuration.

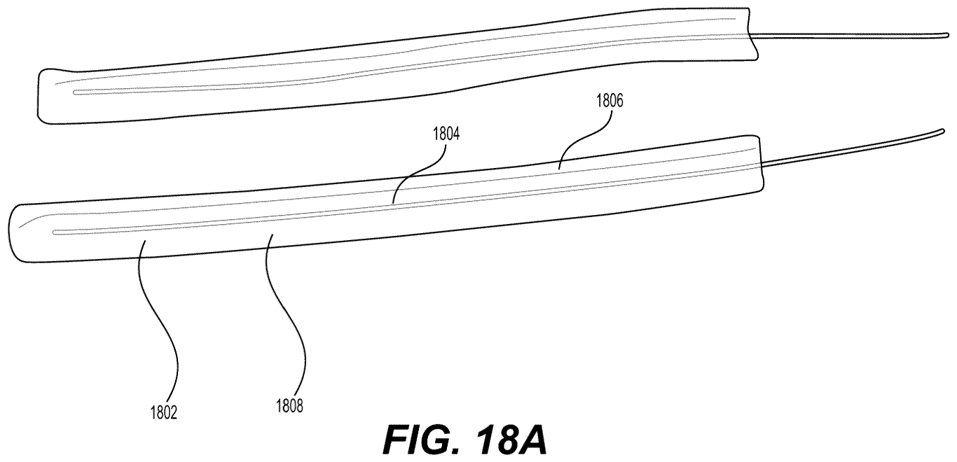

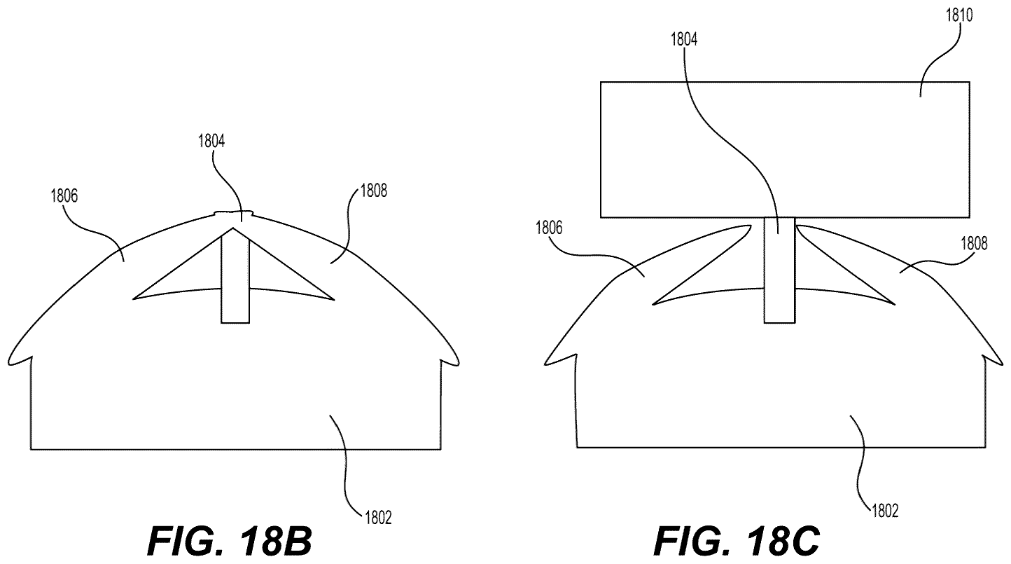

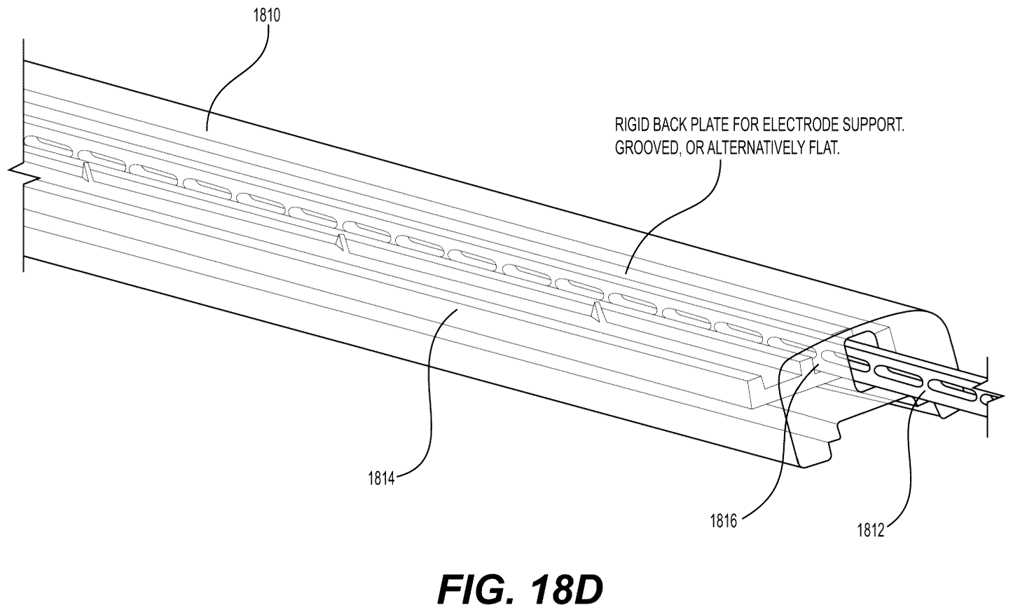

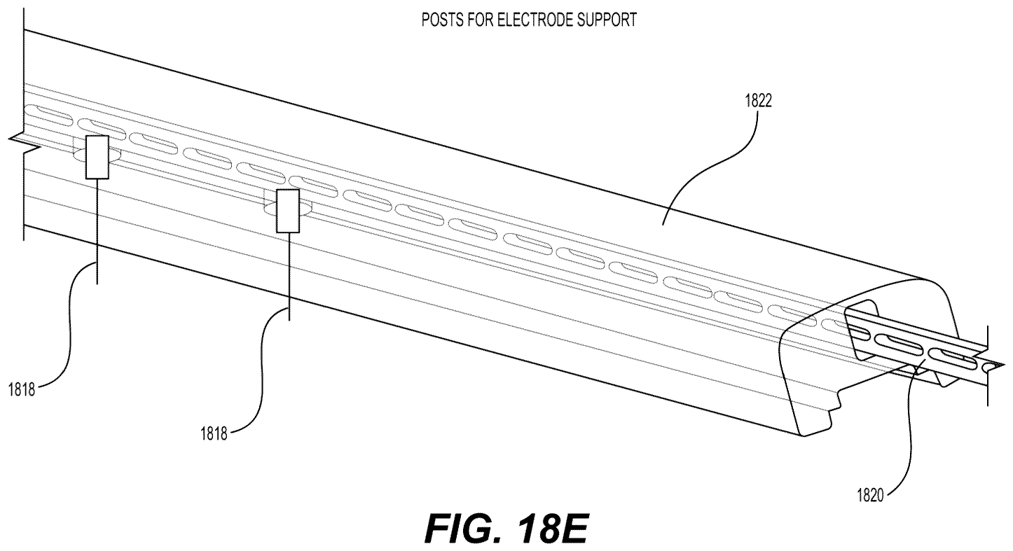

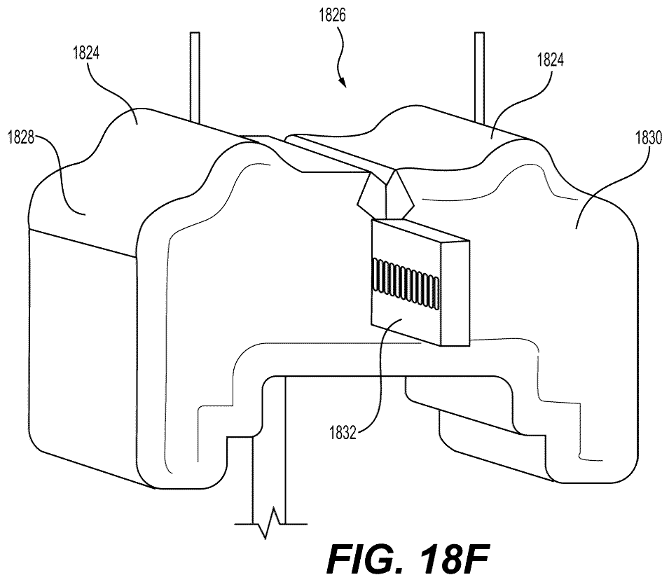

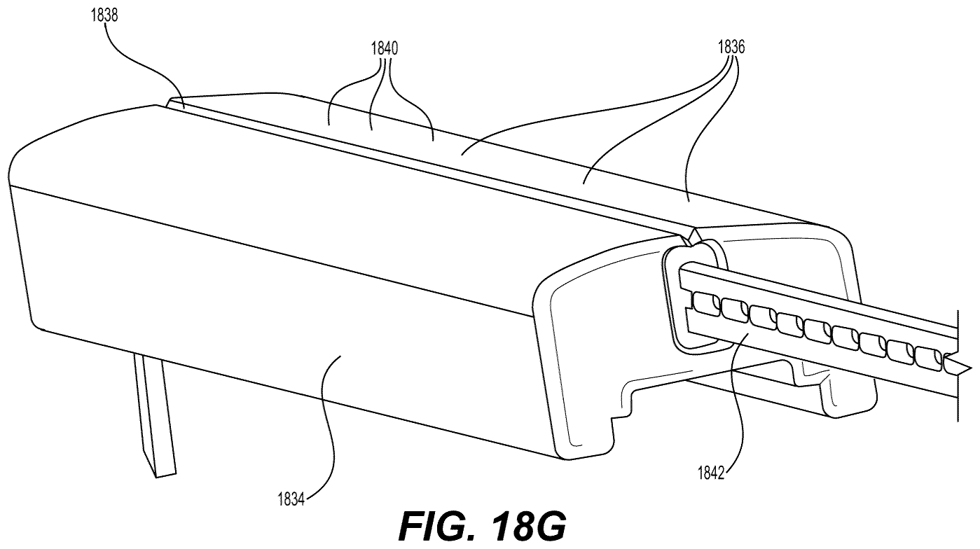

Turning to FIG. 18A, an alternative example configuration comprising a jaw with selectively insulated electrodes is illustrated. FIGS. 18B and 18C are section views of the embodiment of FIG. 18A with the electrodes in contact with the target tissue. In the illustrated embodiment, a jaw 1802 may include one or more electrodes 1804 on a tissue-engaging surface and one or more deformable insulators 1806, 1808, generally similar to the corresponding components described with reference to FIGS. 16A-16C. In the illustrated embodiment, the insulators 1806, 1808 may be at least partially overlapped, such as proximate the electrode 1804. In the illustrated embodiment, a main body of the jaw 1802 may be formed integrally with the insulators 1806, 1808. In some embodiments, the main body of the jaw 1802 may be formed of the same material as the insulators 1806, 1808. Thus, in some embodiments, the main body of the jaw 1802 may be deformable. In some example embodiments, the main body of the jaw 1802 may be mounted to a rigid or more rigid jaw backer when assembled into a clamp-type configuration. In the illustrated embodiments, the configuration of FIGS. 17A-17C also differs from the configuration of FIGS. 18A-18C in that jaw 1702 may be generally longitudinally curved, while jaw 1802 may be generally longitudinally straight.

FIG. 18D illustrates an example embodiment including a deformable insulator 1810 disposed about an electrode 1812 disposed on a rigid back plate 1814. In the illustrated embodiment, the back plate may include one or more longitudinal grooves 1816, such as to receive the electrode therein. In some alternative embodiments, the back plate 1814 may be generally flat (e.g., without grooves).

FIG. 18E illustrates an example embodiment including spaced-apart posts 1818 supporting an electrode 1820 within a deformable insulator 1822. The posts 1818 may be mechanically coupled to a relatively rigid portion support element, such a jaw structure of a clamp-type PFA device. In some embodiments, the insulator 1822 may include one or more openings configured to receive the posts 1818 therethrough.

Some example embodiments may include one or more leading, raised features configured to facilitate movement of insulator material, such as to expose an electrode. For example, as illustrated in FIG. 18F, one or more raised features 1824 on a tissue-contacting surface 1826. These features may be arranged to contact a target tissue before other portions of the insulators 1828, 1830, thus causing the insulators 1828, 1830 to move away from and exposing the electrode 1832.

FIG. 18G illustrates an alternative example embodiment in which a deformable insulator 1834 may be segmented. In the illustrated embodiment, one or more generally lateral cross cuts 1836 (e.g., slits) are provided in addition to a longitudinal slit 1838. Accordingly, segments 1840 of the insulator 1834 in contact with tissue may more easily move away from the electrode 1842, while segments 1840 of the insulator 1834 not in contact with tissue may remain in place (e.g., at least partially covering the electrode 1842).

The present disclosure contemplates that microbubbles may be formed when high-voltage electrical pulses are delivered during PFA. Generally, microbubbles may include a plurality of thin spheres of liquid respectively encapsulating a small pocket of gas. Microbubbles may be formed by liquid vaporization, cavitation, and/or electrolysis, for example. Generally, microbubble formation may be undesirable because, after being formed, the microbubbles may travel through the bloodstream and block small blood vessels, potentially leading to unintended tissue damage and/or organ dysfunction. Microbubbles may also cause silent cerebral events, such as brain injuries that occur during a medical procedure or intervention without producing any noticeable symptoms.

The present disclosure contemplates that although PFA is generally considered non-thermal in that it does not rely on high temperatures to ablate tissue, in some circumstances, application of PFA energy may result in tissue heating. Generally, the duration, intensity, and/or frequency of the PFA signal may affect the extent of tissue heating. In some circumstances, the composition and/or structure of the tissue may affect heating, such as how quickly heat may be transmitted and dissipated. In some circumstances, cooling methods may be utilized to reduce tissue temperature. For example, irrigation with a cooling fluid such as saline and/or use of a heat sink or cooling catheter may reduce the likelihood of undesired heating. Alternatively, in vacuum pod embodiments, fluid flow through the vacuum pod may be employed to cool the electrode and tissue surface. Electrode pair alternating switching may be employed for on/off electrode duty cycling.

The present disclosure contemplates that PFA and RF ablation may be associated with different mechanisms of action and/or potentially different advantages and/or disadvantages. In some example embodiments according to at least some aspects of the present disclosure, these differences may be utilized to facilitate a desired outcome. For example, some illustrative embodiments may be configured to conduct both PFA and RF ablation on a particular target tissue. In some example embodiments, PFA and RF ablation may be performed using at least one electrode in common. In some example embodiments PFA and RF ablation may be performed using different electrodes. In general, any system, device, electrode, insulation configuration, etc., described herein may be used in connection with delivery of either or both RF and PFA.

For example, some embodiments may be user-selectable between PFA-only and RF-only modes. Accordingly, a user may select a desired ablation modality for a particular ablation. For example, a surgeon may select PFA and/or RF ablation based at least in part upon the location of the ablation (e.g., proximity to sensitive, non-target tissues) and/or the target tissue type.

Some example embodiments may be configured to create lesions using both PFA and RF ablation modalities. For example, in some circumstances, it may be advantageous to perform a PFA endocardially in connection with an epicardial RF ablation. The resulting ablation may be partially PFA and partially RF at any mixed ratio meeting within the tissue thickness to create a full thickness lesion. PFA may extend from one surface into the tissue thickness while RF extends from the opposite surface. Alternatively, the RF lesion may partially or wholly be formed centrally within the thickness of the tissue with PF completing the lesion outward to the tissue surfaces. Such a mixed modality approach may create a transmural lesion in the target tissue while benefiting from advantages associated with each individual modality. For example, in some circumstances, using PFA endocardially may avoid some potential disadvantages of using RF ablation in close proximity to blood and/or using RF ablation epicardially may facilitate thermal ablation of some targeted autonomic nervous tissues. In some example embodiments, PFA may be applied bidirectionally (e.g., endocardially and epicardially), and RF ablation may be applied unidirectionally (e.g., epicardially only). In some example embodiments, both PFA and RF ablation may be conducted substantially across a full tissue thickness.

In some example embodiments, PFA may be conducted before RF ablation, which may facilitate faster RF ablation due to the increased tissue conductivity caused by the PFA. In some example embodiments, RF ablation may be conducted before PFA. In some example embodiments, the RF and PF ablations may be performed simultaneously with interrupted alternating delivery and/or overlapping delivery.

The present disclosure contemplates that PFA and cryoablation may be associated with different mechanisms of action and/or different advantages and disadvantages. In some example embodiments according to at least some aspects of the present disclosure, these differences may be utilized to facilitate a desired outcome. For example, some illustrative embodiments may be configured to conduct both PFA and cryoablation, not necessarily at the same time.

For example, some embodiments may be user-selectable between PFA-only and cryoablation-only modes. Accordingly, a user may select a desired ablation modality for a particular ablation. For example, a surgeon may select PFA and/or cryoablation based at least in part upon the location of the ablation and/or the target tissue type.

Some example embodiments may be configured to create lesions using both PFA and cryoablation modalities. Such a mixed modality approach may create a transmural lesion in the target tissue while benefiting from advantages associated with each individual modality. In some example embodiments, PFA may be conducted before cryoablation. In some example embodiments, cryoablation may be conducted before PFA. In some example embodiments, PFA may be conducted during cryoablation at any point in the cryo delivery or for the entire duration. A temperature measurement or setpoint may or may not be employed as feedback. In some example embodiments, the cryoablation may be applied at a therapeutic level. That is, the cryoablation, itself, may be sufficient to cause permanent lesion formation in the target tissue. In other embodiments, the cryoablation may be applied at a sub-therapeutic level. That is, the cryoablation, itself, may affect the tissue in a substantially reversible manner. In some circumstances, performing cryoablation before PFA may affect (e.g., improve or enhance) the subsequent PFA. For example, cryo-treated target tissue either fully rewarmed or still cooled below body temperature may more efficiently receive PFA energy, or the PFA energy may be conducted through the target tissue differently, or the lower tissue temperature effectuated using cryoablation may offset or reduce the thermal temperature increase effectuated from using PFA.

The present disclosure contemplates that, in some circumstances, a PFA-created lesion may not be readily visible on the target tissue immediately or shortly after delivery of the PFA energy. In some cases, a PFA lesion may not be readily visible or detectable for days to weeks, as cell death and tissue response occurs. Accordingly, the existence, location, and/or extent of a PFA lesion may not be readily apparent to a user or detectable during an ablation procedure. The present disclosure contemplates that this lack of immediate visibility and/or detectability may increase the difficulty of creating elongated, continuous lesions formed by conducting multiple, overlapping ablations.

Turning back to FIG. 1, in some example embodiments according to at least some aspects of the present disclosure, a PFA device 104 may be operated to create an immediately visible, thermal lesion in connection with creating a PFA lesion. For example, a PFA lesion may be formed and then a corresponding thermal lesion may be formed using RF ablation and/or cryoablation (e.g., without moving the end effector 132 between the PFA and RF or cryo ablation). Alternatively, high-voltage PFA may be utilized to create a localized thermal lesion in addition to ablating the surrounding tissue via irreversible electroporation. Alternatively, PFA may be conducted, followed by a high-voltage pulse or pulse train that may not electrically porate the tissue but may create a thermal lesion in the tissue. In some example embodiments, a thermally induced lesion may be advantageous as it may ablate surface plexuses.

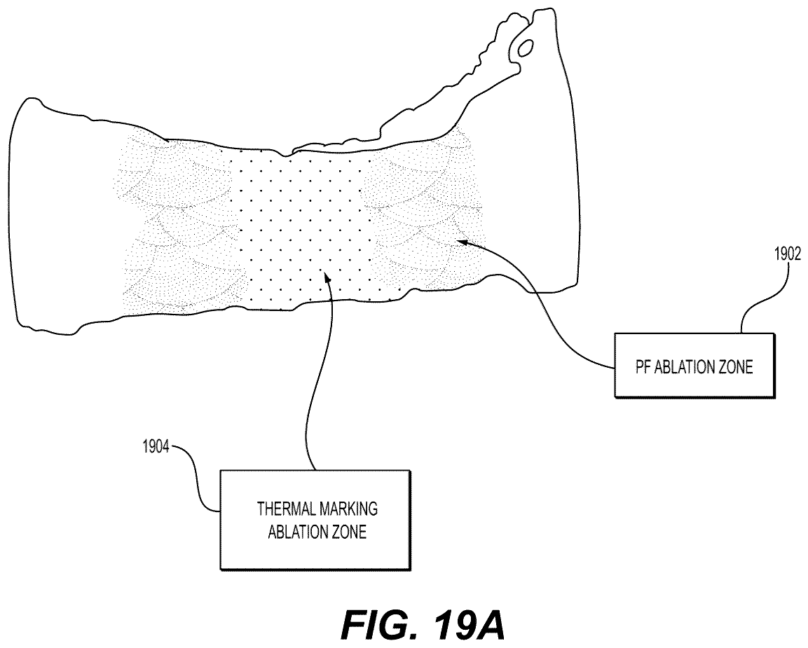

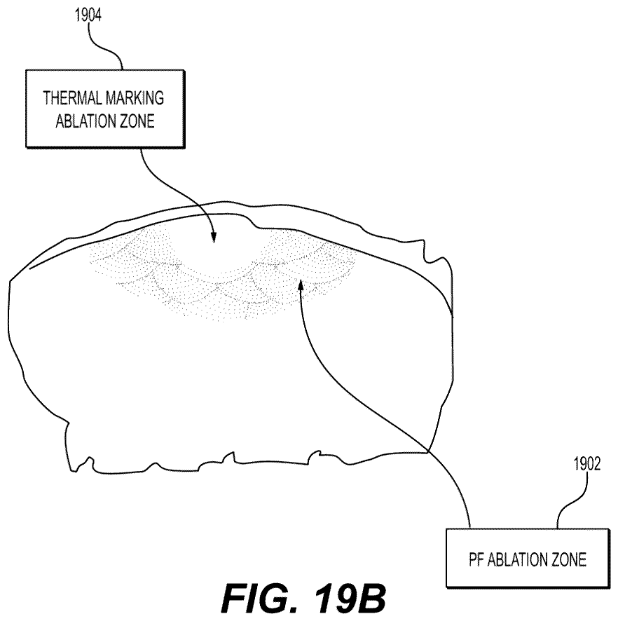

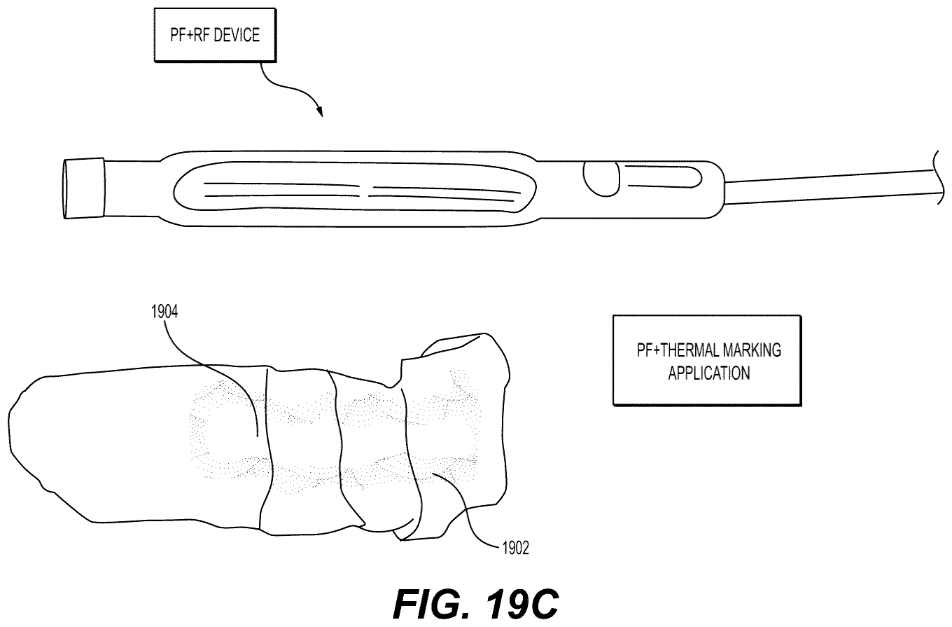

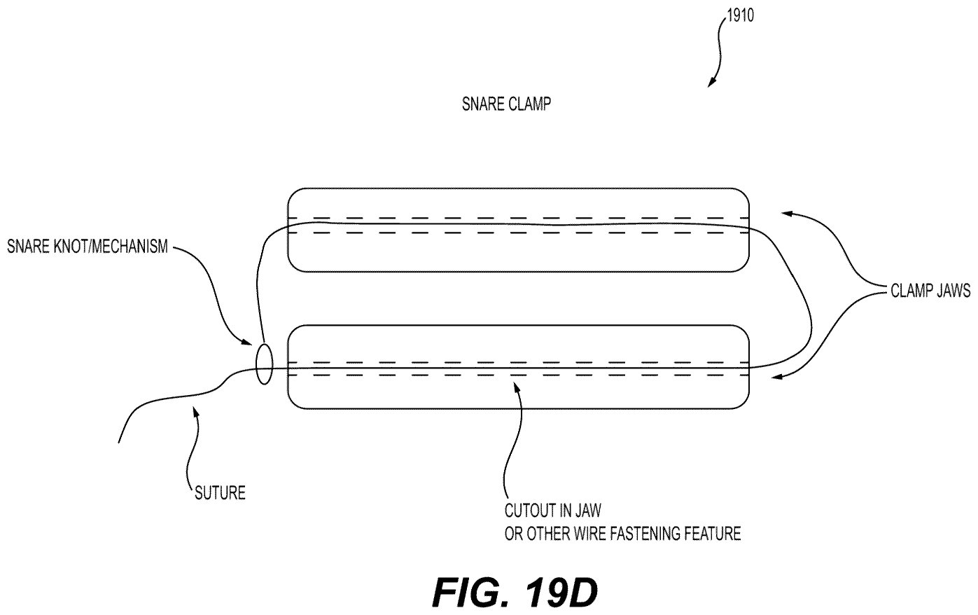

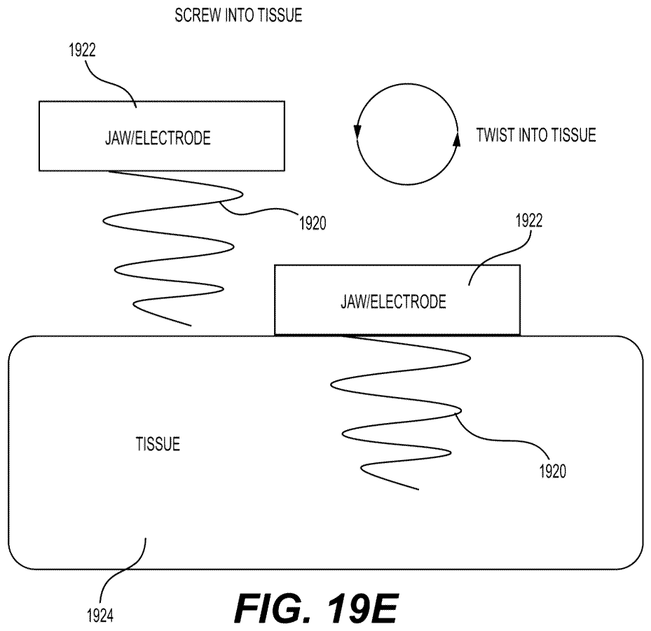

Referencing FIG. 19A, an example lesion including a PFA zone 1902 and a thermal ablation zone 1904 is depicted. FIG. 19B is a section view of the lesion of FIG. 19A.

FIG. 19C is a top view of an example lesion created using a PFA+RF device, as shown having a PFA zone 1902 and a thermal ablation zone 1904, according to at least some aspects of the present disclosure.

Example embodiments according to at least some aspects of the present disclosure may utilize various tissue contact configurations.

Some example embodiments may be configured to utilize operator-applied contact forces to cause the desired tissue contact. For example, pen-type configurations may be manually held against the target tissue by an operator during application of PFA energy. Similarly, a surgical robot may be used to apply a PFA device against a target tissue.

Some embodiments utilizing mechanical configurations may be constructed generally in the form of a clamp. See, for example, the embodiment of FIG. 2. Some embodiments utilizing mechanical configurations may utilize a snare. FIG. 19D illustrates an example snare clamp 1910.

Some example embodiments may utilize a screw for tissue engagement. FIG. 19E illustrates an example generally helical screw engagement element 1920 for a jaw or electrode 1922 configured to penetrate the target tissue 1924.

Some example embodiments may utilize cooperating magnetic elements for tissue engagement. For example, various embodiments described in International Application No. PCT/US2022/082057, filed Dec. 20, 2022, published as International Publication No. WO2023129842 on Jul. 6, 2023, titled “MAGNETICALLY COUPLED ABLATION COMPONENTS”, which is incorporated herein by reference, may be utilized in connection with embodiments according to at least some aspects of the present disclosure.

Some example embodiments may be configured to pierce tissue. See, for example, the needle-type embodiments of FIGS. 4A-4C.

Some example embodiments may be configured to utilize vacuum for tissue engagement. See, for example, the embodiments of FIGS. 3A and 3B.

Some example embodiments may utilize tissue freezing for tissue engagement. For example, an embodiment including cryogenic capabilities may be placed into contact with a target tissue. The tissue may be at least partially frozen or significantly cooled, therapeutically or sub-therapeutically, which may cause a tissue engaging probe to adhere to the target tissue. While the probe is adhered to the target tissue, thus maintaining contact, PFA energy may be applied. After the desired PFA and/or cryogenic effects have been achieved, the tissue may be thawed or heated and the probe may be removed from the tissue.

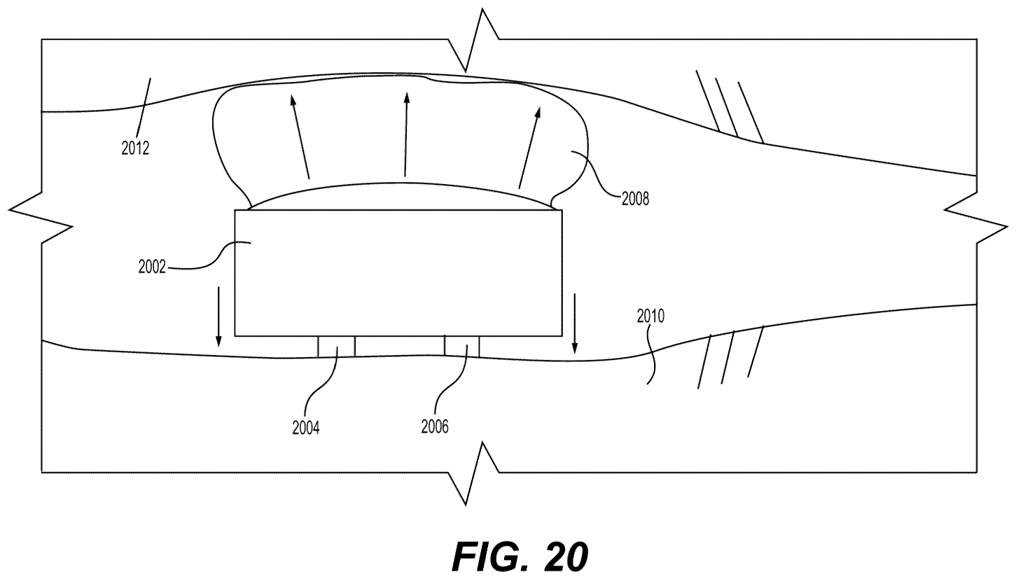

Referring to FIG. 20, some example embodiments may utilize one or more expandable structures to generate a tissue contacting force. In the illustrated embodiment, a PFA device 2002 may include one or more electrodes 2004, 2006 on a tissue-engaging surface and/or one or more expandable structures 2008. In the illustrated embodiment, the expandable structure 2008 may be disposed generally opposite the electrodes 2004, 2006. In operation, the PFA device 2002 may be positioned between a target tissue 2010 and an opposed tissue 2012. The expandable structure 2008 may be expanded from a collapsed configuration (dashed line) to an expanded configuration (solid line). Expanding the expandable structure 2008 may cause it to contact the opposed tissue 2012, thereby pressing the tissue-engaging surface comprising the electrodes 2004, 2006 against the target tissue 2010. Expansion of the expandable structure 2008 may be controlled as necessary to achieve the desired contact between the electrodes 2004, 2006 and the target tissue 2010. Upon completion of one or more ablations, the expandable structure 2008 may be collapsed. In some example embodiments, the expandable structure may be inflatable, such as by a fluid (e.g., gas and/or liquid). In an example embodiment, the target tissue 2010 may comprise a myocardium and the opposed tissue 2012 may comprise a pericardium.

Some example embodiments according to at least some aspects of the present disclosure may include a plurality of electrodes. Generally, any embodiment described herein may be provided with one or multiple electrodes, unless explicitly stated otherwise. While some embodiments may have been described in connection with particular exemplary uses of particular individual electrodes, it is to be understood that any electrode on any embodiment may be used for any purpose, regardless of how it may be described in a specific example. For example, an electrode described as an ablation electrode may be used for pacing, stimulating, mapping, and/or sensing in some circumstances. Similarly, an electrode described as a pacing, stimulating, mapping, and/or sensing electrode may be used for ablation in certain circumstances. Further, whether or not specifically described herein in connection with a particular example embodiment, it is to be understood that any embodiment according to at least some aspects of the present disclosure may include additional electrodes, such as for pacing, stimulating, mapping, and/or sensing.

Some example embodiments according to at least some aspects of the present disclosure may be used in connection with procedures directed to treatment of various arrythmias. For example, ablations may be performed on various target tissues comprising the cardiac autonomic nervous system (e.g., ganglionated plexuses, nodes, and/or conduction pathways) and/or cardiac substrate tissues (e.g., atria and/or ventricles).

Generally, it is within the scope of this disclosure to conduct procedures involving any portions of the heart using apparatus and/or methods disclosed herein. For example, procedures involving the right atrium may be performed in connection with treatment for inappropriate sinus tachycardia (e.g., crista line, inferior vena cava, and/or superior vena cava), atrial fibrillation (e.g., Cox maze lesions—right side), and/or Wolff-Parkinson-White Syndrome. Procedures involving the right ventricle may be performed in connection with treatment for ventricular tachycardia (e.g., right ventricle posterior wall, right ventricle lateral free wall, right ventricle anterior, septum, right ventricle papillary muscles, and/or right ventricle outflow tract), partial ventricular contractors (e.g., right ventricle outflow tract septum, basal right ventricle, and/or right ventricle outflow tract free wall), and/or Brugada Syndrome (e.g., right ventricle outflow tract), for example. Procedures involving the left atrium may be performed in connection with treatment for atrial fibrillation (e.g., ligament of Marshall, roof and floor lines, left atrium posterior wall, isthmus line, and/or autonomics (ganglionated plexus)) and/or left atrial appendage isolation (e.g., left atrial appendage ostium). Procedures involving the left ventricle may be performed in connection with syncope (e.g., autonomics (ganglionated plexus)), atrial tachycardia (e.g., anywhere in the left ventricle), atrial flutter (e.g., mitral valve), Wolff-Parkinson-White Syndrome (e.g., atrioventricular groove), partial ventricular contractions (e.g., left ventricle outflow tract and/or aortic root), hypertension (e.g., anywhere in the left ventricle), and/or ventricular tachycardia (e.g., left ventricle posterior wall, left ventricle lateral free wall, left ventricle anterior, septum, left ventricle papillary muscles, and/or left ventricle summit), for example. Procedures involving the right ventricle/left ventricle septum may be performed in connection with ventricular tachycardia (e.g., combined right ventricle and left ventricle lesion), for example. It will be understood that the foregoing list is merely exemplary and is not to be considered limiting.

Some example embodiments according to at least some aspects of the present disclosure may be used in connection with nerve block procedures. For example, peripheral nerves may be ablated to create a temporary yet fully recoverable loss of sensory nerve function. Ablation may cause axonotmesis, a level of nerve injury according to Seddon's classification in which the axons and the myelin are disrupted but at least some of the surrounding tubular structures, such as the endoneurium, perineurium, fascicle, and/or epineurium, remain intact. The ensuing Wallerian degeneration, a process in which the entire length of the nerve segment distal to the ablation lesion is dismantled, may take approximately 1 week. Regeneration of the nerve begins from the proximal segment and continues at an average rate of 1-3 mm/day, following the intact structural components until the tissue is reinnervated. This process can take weeks to months depending on how significant the ablation lesion is on the tissue. Because it preserves the structure of the nerve, such procedures may not be associated with development of neuromas.

Local analgesia to a nerve (e.g., intercostal nerve) is intended for managing pain due to incision, surgical muscle disruption, discomfort from nerve impingement by the surgical equipment (e.g., retractors) and surgical retainers (e.g., sutures), and for any opening created by a tube or trocar site. In exemplary form, one exemplary process comprises nerve ablation for post-thoracotomy pain that includes ablation of the intercostal nerves. What follows is an exemplary procedure for conducting a nerve block responsive to a thoracotomy that is effective for pain management and may be applied to any nerve within an animal body.

It may be recommended to perform the nerve ablation procedure as early as possible in the surgical procedure, such as prior to or immediately following creation of the thoracotomy. The target nerve, such as an intercostal nerve, may be located in the incisional intercostal space (e.g., between the ribs), preferably at the margin of the innermost intercostal muscle and the membranous portion of the internal intercostal muscle. A location may be chosen that is proximal to the lateral cutaneous branch but at least 2 cm from the ganglia and/or at least 4 cm from the spine.

The ablation device may be placed directly on top of the nerve, optionally with a slight angulation that assures the nerve is directly under the ablation element. Prior to ablation, the ablation device may be pressed into the costal groove with enough pressure to create compression of the tissue for stability and reduced local perfusion. Adequate pressure may be pressure sufficient to create blanching if depressed against the skin. In some example embodiments, a needle-type PFA device may be used. In some example embodiments, a pen-type PFA device may be used. In some example embodiments, a minimally invasive PFA device may be used, such as one providing vacuum stabilization capability.

Post locating the ablation device to contact the nerve or in close proximity thereto, the ablation device may be activated to ablate the nerve. The ablation sequence may be repeated at another location of the same nerve (or at a different location of a different nerve) and repeated as necessary to achieve the proper pain management result. In general, some exemplary nerve ablation procedures as described above may be repeated on the intercostal nerves located in each of the third to ninth intercostal spaces.

In some example methods according to at least some aspects of the present disclosure, nerve ablation may be provided in connection with amputation of a limb and/or an extremity. The present disclosure contemplates that, in some circumstances, nerve ablation may be performed at some time after an amputation procedure is performed (e.g., weeks, months, or years), such as after the occurrence of significant pain for the patient.

In some example methods according to at least some aspects of the present disclosure, nerve ablation may be performed concomitant with the amputation procedure. For example, during an amputation procedure, a nerve may be identified. The nerve may be dissected, separating it from adjacent tissues, such as nearby blood vessels. A nerve section location may be determined. In some cases, the nerve may be retracted distally. A nerve ablation location may be determined, such as proximal to the nerve transection location. The nerve may be engaged at the ablation location, such as using an ablation device. Ablation of the nerve may be performed using the ablation device either by contact with a pen like device or capturing between a clamp like or grasper like device. In some cases, one or more ablation cycles may be performed. The ablation device may be removed from the nerve. The nerve may be sectioned at the nerve section location.

Thus, the mechanical injury to the nerve (e.g., the transection of the nerve at the nerve section location) may be some distance distal to the nerve ablation location. Because the nerve may slowly regenerate distally from the ablation location towards the nerve section location, it may take substantial time until the regenerated nerve reaches the nerve section location. During this time, injured tissues proximate the nerve section location may heal. As a result, when the regenerated nerve reaches the nerve section location, the nerve may be surrounded by relatively healed tissue, thus reducing the likelihood and/or severity of neuroma formation. Additionally, during the time it takes for the nerve to regenerate, pain and other sensations from locations distal to the ablation location may be reduced, thus reducing the need for other post-operative pain management.

Some example embodiments according to at least some aspects of the present disclosure may be utilized in connection with ablation of target tissues other than cardiac tissue and nervous tissue. For example, some embodiments may be used in connection with ablation of tissues including liver tissue, kidney tissue, and/or brain tissue.

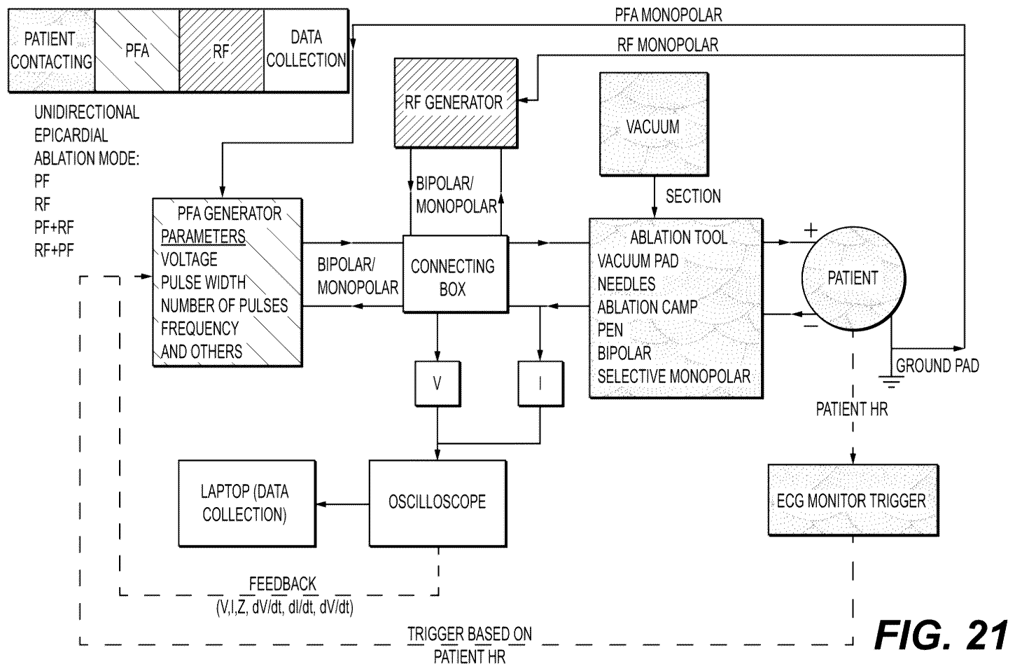

Turning to FIG. 21, a simplified block diagram of an example equipment configuration, which may be used, for example, for using various PFA and/or RF ablation devices and/or algorithms according to at least some aspects of the present disclosure is illustrated.

Referencing FIG. 22, an example PFA device 2200 may be configured for vacuum-stabilization and/or monopolar energy delivery. The illustrated embodiment may be constructed and/or operated generally similar to those described in U.S. Pat. No. 10,413,355, issued Sep. 17, 2019, titled “VACUUM COAGULATION PROBES,” which is incorporated by reference herein. Referring to FIGS. 5R and 22, some example embodiments may include a generally helical electrode 2202 disposed within a vacuum pod, which may engage the target tissue using vacuum and/or which may be supplied with saline solution, which may facilitate cooling and/or coupling).

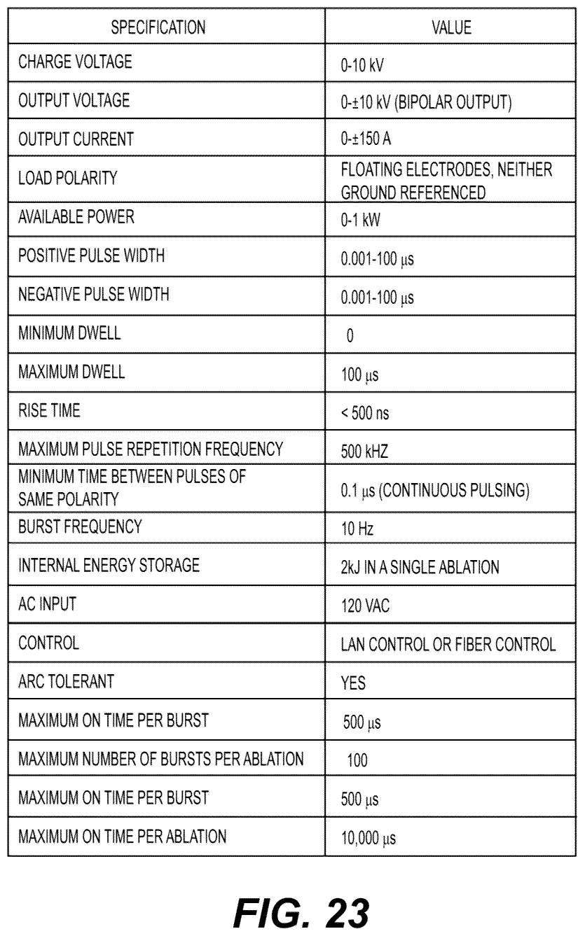

Referring to FIG. 23, in some example embodiments according to at least some aspects of the present disclosure, one or more PFA energy parameters, such as those described herein, may be varied based at least in part upon one or more measured parameters. For example, prior to delivering PFA energy, one or more measurements may be conducted, and such measured values may be used to determine one or more PFA energy parameters that will be delivered. In some example embodiments, one or more measurements may be conducted in connection with delivery of PFA energy (e.g., during and/or between PFA pulses), and such measured values may be used to determine whether to continue or stop PFA energy delivery and/or may be used to determine whether to adjust one or more PFA energy parameters.

In some embodiments involving a clamp-type PFA device, one or more parameters associated with the distance between the jaws may be used to determine, at least in part, one or more PFA energy parameters. For example, one or more PFA energy parameters may be controlled as a function of a parameter associated with a distance between opposed jaws. For example, in one embodiment, the distance between the jaws may be measured and may be used to determine the PFA energy potential (e.g., maximum voltage delivered). In some such embodiments, a greater measured jaw distance may result in a greater PFA energy potential. Accordingly, in some embodiments, a desired cellular transmembrane voltage potential may be achieved across a range of jaw closure distances.

In some example embodiments, at least one PFA energy parameter may increase as a measured parameter increases. In some example embodiments, at least one PFA energy parameter may decrease as a measured parameter decreases. In some example embodiments, at least one PFA energy parameter may increase as a measured parameter decreases. In some example embodiments, at least one PFA energy parameter may decrease as a measured parameter increases.

In some example embodiments, at least one PFA energy parameter may vary substantially linearly with a measured parameter. In some example embodiments, at least one PFA energy parameter may vary substantially non-linearly with a measured parameter.

For example and without limitation, one or more PFA energy parameters, such as those described herein, may be determined and/or may be varied based at least in part upon one or more measurements of voltage, current, inductance, impedance, conductivity, resistance, or temperature.

In some example embodiments according to at least some aspects of the present disclosure, one or more PFA energy parameters, such as those described herein, may be varied based at least in part upon one or more selected parameters. Such selected parameters may be preset when a device or unit is constructed for a particular end use, or may be selected by a user before and/or during use. For example, tissue type, cell density, and/or tissue compression/compressibility may be used to determine, at least in part, one or more PFA energy parameters.

In some example embodiments, two or more measured and/or selected parameters may be used in combination to determine one or more PFA energy parameters. For example, a selected tissue type (which may be associated with known tissue compressibility and/or cell density values, for example), a measured jaw closure distance, and/or a measured jaw closure force may be used, in combination, to determine, at least in part, at least one PFA energy parameter, such as maximum potential. In some embodiments, two or more measured and/or selected parameters may be equally weighted in determining at least one PFA energy parameter. In some embodiments, two or more measured and/or selected parameters may be unequally weighted in determining at least one PFA energy parameter. In some example embodiments, different selected and/or measured parameters may be used to determine, at least in part, different PFA energy parameters. In some example embodiments, different selected and/or measured parameters may be weighted differently in connection with determining, at least in part, different PFA energy parameters.

Generally, any one or more PFA energy parameters described herein may be determined and/or varied based at least in part upon selection and/or measurement of any parameter or condition described herein.

The following patent references may provide context for the present disclosure and are incorporated by reference herein in their entireties: U.S. Pat. No. 9,072,518, issued Jul. 7, 2015, titled “HIGH-VOLTAGE PULSE ABLATION SYSTEMS AND METHODS”; U.S. Pat. No. 9,474,574, issued Oct. 25, 2016, titled “STABILIZED ABLATION SYSTEMS AND METHODS”; U.S. Pat. No. 11,628,007, issued Apr. 18, 2023, titled “CRYOPROBE”; U.S. Pat. No. 10,413,355, issued Sep. 17, 2019, titled “VACUUM COAGULATION PROBES”; U.S. Patent Application Publication No. 2022/0133400, published May 5, 2022, titled “ABLATION DEVICES AND METHODS OF USE”; U.S. Patent Application Publication No. 2019/0159835, published May 30, 2019, titled “CRYOPAD”; and International Application No. PCT/US2022/082057, filed Dec. 20, 2022, published as International Publication No. WO2023129842 on Jul. 6, 2023, titled “MAGNETICALLY COUPLED ABLATION COMPONENTS.” Generally, any features or improvements described herein may be used in connection with embodiments described in these patent references, and any features, elements, or methods described in these patent references may be used in connection with any embodiments described herein.

Unless specifically indicated, it will be understood that the description of any structure, function, and/or methodology with respect to any illustrative embodiment herein may apply to any other illustrative embodiments. More generally, it is within the scope of the present disclosure to utilize any one or more features of any one or more example embodiments described herein in connection with any other one or more features of any other one or more other example embodiments described herein. Accordingly, any combination of any of the features or embodiments described herein is within the scope of this disclosure.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A pulsed field ablation effector comprising:
an electrode including an electrode surface for delivering electric current to anatomical tissue; and,
a deformable insulator selectively covering the electrode surface, the deformable insulator configured to deform when contacted by the anatomical tissue to expose the electrode surface.

2. The pulsed field ablation effector of claim 1, wherein the deformable insulator includes a slit at least partially occupied by the electrode.

3. (canceled)

4. The pulsed field ablation effector of claim 1, further comprising a rigid backer to which the electrode and deformable insulator are mounted.

5. (canceled)

6. (canceled)

7. The pulsed field ablation effector of claim 1, wherein the deformable insulator includes a raised feature configured to concentrate contact force from contact with the anatomical tissue and hasten deformation of the deformable insulator.

8. (canceled)

9. (canceled)

10. The pulsed field ablation effector of claim 1, wherein the deformable insulator comprises an elastomer.

11. (canceled)

12. The pulsed field ablation effector of claim 1, wherein:
the electrode is segmented into a plurality of electrodes;
the deformable insulator is segmented into a plurality of deformable insulator sections;
the plurality of electrodes each include electrode surfaces that are selectively covered by at least one of the plurality of deformable insulator sections; and,
only those of the plurality of deformable insulator sections contacted by the anatomical tissue deform to expose those of the plurality of electrodes covered by the plurality of deformable insulator sections contacted.

13. The pulsed field ablation effector of claim 1, wherein:
the deformable insulator is segmented into a plurality of deformable insulator sections;
the electrode surface is selectively covered by at least one of the plurality of deformable insulator sections; and,
only those of the plurality of deformable insulator sections contacted by the anatomical tissue deform to expose those aspects of the electrode surface covered by the plurality of deformable insulator sections contacted.

14. The pulsed field ablation effector of claim 1, further comprising a cryogenic conduit configured to supply cryogenic fluid to a cryogenic tissue contact.

15. (canceled)

16. The pulsed field ablation effector of claim 1, further comprising a radio frequency electrode adapted to deliver radio frequency energy to the anatomical tissue.

17. (canceled)

18. A method of performing a pulsed field tissue ablation, the method comprising:
repositioning a pulsed field ablation effector into proximity with a target tissue, where the pulsed field ablation effector includes an electrode having an ablation surface covered by a deformable insulator;
repositioning the pulsed field ablation effector to sufficiently contact the target tissue, where sufficient contact with the target tissue is operative to deform the deformable insulator and expose an ablation surface of the electrode that was previously covered by the deformable insulator;
supplying electric current to the electrode, when in sufficient contact with the target tissue, to cause electroporation to the target tissue; and,
repositioning the pulsed field ablation effector to no longer sufficiently contact the target tissue, where insufficient contact with the target tissue is operative to deform the deformable insulator and cover the ablation surface of the electrode that was previously uncovered.

19. The method of claim 18, wherein:
the target tissue is cardiac tissue; and,
the contact is epicardial contact.

20. (canceled)

21. (canceled)

22. The method of claim 18, wherein:
the target tissue is cardiac tissue; and,
the contact is endocardial contact.

23. The method of claim 18, wherein:
the pulsed field ablation effector includes a first jaw and a second jaw, where the electrode includes a first electrode portion on the first jaw and a second electrode portion on the second jaw; and,
repositioning the pulsed field ablation effector to sufficiently contact the target tissue includes bringing the first electrode portion into contact with epicardial cardiac tissue, and bringing the second electrode portion into contact with endocardial cardiac tissue, so that sufficient contact with the epicardial cardiac tissue and endocardial cardiac tissue is operative to deform the deformable insulator and expose the first and second electrode portions previously covered by the deformable insulator.

24. The method of claim 18, further comprising:
conducting a cryoablation concomitant with the electroporation to cause destruction of the target tissue or tissue in proximity thereto.

25. The method of claim 18, further comprising:
conducting a radio frequency ablation concomitant with the electroporation to cause destruction of the target tissue or tissue in proximity thereto.

26. The method of claim 18, wherein:
the deformable insulator interposes the ablation surface of the electrode and the target tissue in the absence of sufficiently contact between the target tissue and the deformable insulator; and,
the deformable insulator no longer interposes the ablation surface of the electrode and the target tissue when sufficient contact occurs between the target tissue and the deformable insulator.

27. The method of claim 26, wherein the tissue contacting surface of the electrode erupts from within the deformable insulator upon sufficient contact between the target tissue and the deformable insulator.

28. The method of claim 26, wherein:
the electrode is segmented into a plurality of electrodes, each of the plurality of electrodes having a tissue contacting surface;
the deformable insulator is segmented into a plurality of deformable insulator sections, with each of the plurality of electrodes being selectively covered by at least one of the plurality of deformable insulator sections; and,
each of the plurality of deformable insulator sections is operative to expose the corresponding tissue contacting surface of the plurality of electrodes when sufficient contact occurs between the target tissue and each of the plurality of deformable insulator sections.

29. The method of claim 18, wherein:
the deformable insulator is segmented into a plurality of deformable insulator sections with the ablation surface being selectively covered by at least one of the plurality of deformable insulator sections;
the deformable insulator interposes the ablation surface of the electrode and the target tissue in the absence of sufficiently contact between the target tissue and the deformable insulator; and,
each of the plurality of deformable insulator sections is operative to expose a portion of the ablation surface when sufficient contact occurs between the target tissue and each of the plurality of deformable insulator sections.

30. A method of inhibiting unintended arcing across a pulsed field ablation electrode, the method comprising:
covering the pulsed field ablation electrode with a deformable insulator, the deformable insulator configured to change its shape between a first shape and a second shape responsive to a sufficient external force being applied thereto, the first shape covering a tissue contacting surface of the pulsed field ablation electrode, and the second shape uncovering the tissue contacting surface of the pulsed field ablation electrode.
